(19) United States
(12) Patent Application Publication
(10) Pub. No.: US 2026/0364107 A1
(43) Pub. Date: Nov. 19, 2026

(54) TREATING CANCER

(71) Applicant: Mayo Foundation for Medical Education and Research, Rochester, MN (US)

(72) Inventors: Saad J. Kenderian, Rochester, MN (US); Carli M. Stewart, Rochester, MN (US)

(21) Appl. No.: 19/126,325

(22) PCT Filed: Nov. 1, 2023
(86) PCT No.: PCT/US2023/036570

§ 371 Date: May 1, 2025

Related U.S. Application Data
(60) Provisional application No. 63/421,376, filed on Nov. 1, 2022.

(51) Int. Cl. A61K 35/17; A61K 39/00; A61K 40/11; A61K 40/31; A61K 40/42; A61K 45/06; A61P 35/00; C07K 16/24; C07K 16/28

(52) U.S. Cl. CPC A61K 35/17; A61K 40/11; A61K 40/31; A61K 40/4211; A61K 45/06; A61P 35/00; C07K 16/247; C07K 16/2803; A61K 2039/505; A61K 2239/13; A61K 2239/38; A61K 2239/48

(57) ABSTRACT

This document relates to methods and materials involved in treating cancer. For example, methods and materials for using chimeric antigen receptor (CAR) T cells having reduced levels of an interleukin (IL) 4 polypeptide, having reduced levels of a transcription factor 7 (TCF7) polypeptide, having reduced levels of a protein tyrosine phosphatase non-receptor type 2 (PTPN2) polypeptide, and/or having reduced levels of a protein tyrosine phosphatase non-receptor type 3 (PTPN3) polypeptide are provided. Methods and materials for using such CAR T cells in an adoptive cell therapy (e.g., a CAR T cell therapy) to treat a mammal (e.g., a human) having cancer are also provided.

### CROSS-REFERENCE TO RELATED APPLICATIONS

This application claims the benefit of U.S. Patent Application Ser. No. 63/421,376, filed on Nov. 1, 2022. The disclosure of the prior application is considered part of, and is incorporated by reference in, the disclosure of this application.

### SEQUENCE LISTING

This application contains a Sequence Listing that has been submitted electronically as an XML file named “07039-2171WO1.xml.” The XML file, created on Nov. 1, 2023, is 15,000 bytes in size. The material in the XML file is hereby incorporated by reference in its entirety.

### TECHNICAL FIELD

This document relates to methods and materials involved in treating cancer. For example, this document provides methods and materials for using chimeric antigen receptor (CAR) T cells having reduced levels of an interleukin (IL) 4 polypeptide, having reduced levels of a transcription factor 7 (TCF7) polypeptide, having reduced levels of a protein tyrosine phosphatase non-receptor type 2 (PTPN2) polypeptide, and/or having reduced levels of a protein tyrosine phosphatase non-receptor type 3 (PTPN3) polypeptide. This document also provides methods and materials for using such CAR T cells in an adoptive cell therapy (e.g., a CAR T cell therapy) to treat a mammal (e.g., a human) having cancer.

### BACKGROUND INFORMATION

CAR T cell therapy targeting CD19 (CART19) has shown remarkable overall response rates in the treatment of hematological malignancies. However, durable response rates remain at approximately 40%. CAR T cell in vivo functions depend on their associated cell fate following infusion. Thus, the success of CAR T cell therapies relies on the survival, proliferation, and continued efficacy of the CAR T cells. T cell exhaustion, an acquired state of T cell dysfunction that is associated with decreased proliferation and efficacy, is widely considered a major limitation of CAR T cell therapy.

### SUMMARY

This document provides methods and materials for generating T cells (e.g., CAR T cells) having a reduced level of an IL-4 polypeptide, a reduced level of a TCF7 polypeptide, a reduced level of a PTPN2 polypeptide, and/or a reduced level of a PTPN3 polypeptide. For example, a T cell (e.g., a CAR T cell) can be engineered to have reduced IL-4 polypeptide expression (e.g., for use in adoptive cell). In some cases, a T cell (e.g., a CAR T cell) can be engineered to knock out (KO) KO a nucleic acid encoding an IL-4 polypeptide to reduce IL-4 polypeptide expression in that T cell. In another example, a T cell (e.g., a CAR T cell) can be engineered to have reduced TCF7 polypeptide expression (e.g., for use in adoptive cell therapy). In some cases, a T cell (e.g., a CAR T cell) can be engineered to KO a nucleic acid encoding a TCF7 polypeptide to reduce TCF7 polypeptide expression in that T cell. In another example, a T cell (e.g., a CAR T cell) can be engineered to have reduced PTPN3 polypeptide expression (e.g., for use in adoptive cell therapy). In some cases, a T cell (e.g., a CAR T cell) can be engineered to KO a nucleic acid encoding a PTPN3 polypeptide to reduce PTPN3 polypeptide expression in that T cell.

This document also provides methods and materials for using T cells (e.g., CAR T cells) having a reduced level of an IL-4 polypeptide, a reduced level of a TCF7 polypeptide, a reduced level of a PTPN2 polypeptide, and/or a reduced level of a PTPN3 polypeptide. For example, T cells (e.g., CAR T cells) having a reduced level of an IL-4 polypeptide, a reduced level of a TCF7 polypeptide, a reduced level of a PTPN2 polypeptide, and/or a reduced level of a PTPN3 polypeptide can be administered (e.g., in an adoptive cell therapy) to a mammal (e.g., a human) having cancer to treat the mammal's cancer.

As demonstrated herein, IL-4 polypeptides, TCF7 polypeptides, and PTPN3 polypeptides are mediators of CAR T cell exhaustion. Also as demonstrated herein, CAR T cells having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) are less susceptible to T cell exhaustion, and therefore demonstrate enhanced CAR T cell function and antitumor activity. In some cases, CAR T cells having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) can be incorporated into adoptive T cell therapies (e.g., CAR T cell therapies) to treat, for example, mammals having cancer.

In general, one aspect of this document features a T cell having reduced susceptibility to T cell exhaustion, where the T cell has (a) a reduced level of an interleukin (IL) 4 polypeptide, a reduced level of a transcription factor 7 (TCF7) polypeptide, a reduced level of a protein tyrosine phosphatase non-receptor type 2 (PTPN2) polypeptide or a reduced level of a protein tyrosine phosphatase non-receptor type 3 (PTPN3) polypeptide, where the T cell includes (b) nucleic acid encoding a CAR, and where the T cell expresses the CAR. The T cell can have a reduced level of an TL-4 polypeptide. The T cell having a reduced level of an IL-4 polypeptide can have a disruption in at least one endogenous allele encoding the IL-4 polypeptide. The T cell having a reduced level of an IL-4 polypeptide can have a disruption in both endogenous alleles encoding the IL-4 polypeptide. The T cell having a reduced level of an IL-4 polypeptide can express a reduced level of the IL-4 polypeptide as compared to a comparable T cell lacking the disruption. The T cell can have a reduced level of a TCF7 polypeptide. The T cell having a reduced level of a TCF7 polypeptide can have a disruption in at least one endogenous allele encoding a TCF7 polypeptide. The T cell having a reduced level of a TCF7 polypeptide can have disruption in both endogenous alleles encoding a TCF7 polypeptide. The T cell having a reduced level of a TCF7 polypeptide can express a reduced level of the TCF7 polypeptide as compared to a comparable T cell lacking the disruption. The T cell can have a reduced level of a PTPN2 polypeptide. The T cell having a reduced level of a PTPN2 polypeptide can have a disruption in at least one endogenous allele encoding a PTPN2 polypeptide. The T cell having a reduced level of a PTPN2 polypeptide can have a disruption in both endogenous alleles encoding a PTPN2 polypeptide. The T cell having a reduced level of a PTPN2 polypeptide can express a reduced level of the PTPN2 polypeptide as compared to a comparable T cell lacking the disruption. The T cell can have a reduced level of a PTPN3 polypeptide. The T cell having a reduced level of a PTPN3 polypeptide can have a disruption in at least one endogenous allele encoding a PTPN3 polypeptide. The T cell having a reduced level of a PTPN3 polypeptide can have a disruption in both endogenous alleles encoding a PTPN3 polypeptide. The T cell having a reduced level of a PTPN3 polypeptide can express a reduced level of the PTPN3 polypeptide as compared to a comparable T cell lacking the disruption. The CAR can target a tumor-associated antigen. The tumor-associated antigen can be CD19. The T cell can be obtained from a human. The T cell can exhibit anti-tumor activity for at least 1 week. The T cell can exhibit anti-tumor activity for from about 1 week to about 2 years. The T cell can exhibit proliferation for at least 1 week. In some embodiments, the T cell does not exhibit T cell exhaustion as rapidly as a comparable T cell lacking the reduced level of the IL-4 polypeptide, lacking the reduced level of the TCF7 polypeptide, lacking the reduced level of the PTPN2 polypeptide, and lacking the reduced level of the PTPN3 polypeptide.

In another aspect, this document features methods for treating a mammal having cancer. The methods can include, or consist essentially of, administering, to a mammal having cancer, a composition comprising a T cell having (a) a reduced level of an interleukin (IL) 4 polypeptide, a reduced level of a transcription factor 7 (TCF7) polypeptide, a reduced level of a protein tyrosine phosphatase non-receptor type 2 (PTPN2) polypeptide or a reduced level of a protein tyrosine phosphatase non-receptor type 3 (PTPN3) polypeptide, where the T cell includes (b) nucleic acid encoding a CAR, and where the T cell expresses the CAR. The mammal can be identified as being in need of T cells having reduced susceptibility to CAR T cell exhaustion. The mammal can be a human. The composition can include from about 0.1×10⁶ to about 10×10⁶ of T cells. The cancer can be a lymphoma (e.g., a diffuse large B cell lymphoma). The cancer can be a leukemia (e.g., an acute lymphoblastic leukemia). The CAR can target a tumor-associated antigen. The tumor-associated antigen can be CD19.

In another aspect, this document features uses of a composition including a T cell having (a) a reduced level of an interleukin (IL) 4 polypeptide, a reduced level of a transcription factor 7 (TCF7) polypeptide, a reduced level of a protein tyrosine phosphatase non-receptor type 2 (PTPN2) polypeptide or a reduced level of a protein tyrosine phosphatase non-receptor type 3 (PTPN3) polypeptide, where the T cell includes (b) nucleic acid encoding a CAR, and where the T cell expresses the CAR to treat a mammal having cancer.

In another aspect, this document features compositions including a T cell having (a) a reduced level of an interleukin (IL) 4 polypeptide, a reduced level of a transcription factor 7 (TCF7) polypeptide, a reduced level of a protein tyrosine phosphatase non-receptor type 2 (PTPN2) polypeptide or a reduced level of a protein tyrosine phosphatase non-receptor type 3 (PTPN3) polypeptide, where the T cell includes (b) nucleic acid encoding a CAR, and where the T cell expresses the CAR for use in the preparation of a medicament to treat a mammal having cancer.

In another aspect, this document features compositions including a T cell having (a) a reduced level of an interleukin (IL) 4 polypeptide, a reduced level of a transcription factor 7 (TCF7) polypeptide, a reduced level of a protein tyrosine phosphatase non-receptor type 2 (PTPN2) polypeptide or a reduced level of a protein tyrosine phosphatase non-receptor type 3 (PTPN3) polypeptide, where the T cell includes (b) nucleic acid encoding a CAR, and where the T cell expresses the CAR for use in the treatment of cancer.

In another aspect, this document features methods for providing a mammal with CAR T cells having a reduced susceptibility to T cell exhaustion. The methods can include, or consist essentially of, (a) administering CAR T cells to a mammal, and (b) administering an inhibitor of an IL-4 polypeptide, an inhibitor of a TCF7 polypeptide, or an inhibitor of a PTPN3 polypeptide to the mammal, where the CAR T cells do not exhibit T cell exhaustion within the mammal as rapidly as comparable CAR T cells administered to a comparable mammal not administered the inhibitor of the IL-4 polypeptide, the inhibitor of the TCF7 polypeptide, and the inhibitor of the PTPN3 polypeptide. The mammal can be identified as being in need of T cells having reduced susceptibility to CAR T cell exhaustion. The mammal can be a human. The composition can include from about 0.1×10⁶ to about 10×10⁶ of T cells. The cancer can be a lymphoma (e.g., a diffuse large B cell lymphoma). The cancer can be a leukemia (e.g., an acute lymphoblastic leukemia). The CAR can target a tumor-associated antigen. The tumor-associated antigen can be CD19. The mammal can be administered the inhibitor of the IL-4 polypeptide. The mammal can be administered the inhibitor of the TCF7 polypeptide. The mammal can be administered the inhibitor of the PTPN2 polypeptide. The inhibitor of the PTPN2 polypeptide can be ABBV-CLS-484 or ABBV-CLS-579. The mammal can be administered the inhibitor of the PTPN3 polypeptide. The CAR T cells can include a reduced level of an IL 4 polypeptide, a reduced level of a TCF7 polypeptide, a reduced level of a PTPN2 polypeptide, or a reduced level of a PTPN3 polypeptide. The CAR T cells can have a reduced level of an IL-4 polypeptide. The CAR T cells having a reduced level of an IL-4 polypeptide can have a disruption in at least one endogenous allele encoding the IL-4 polypeptide. The CAR T cells having a reduced level of an IL-4 polypeptide can have a disruption in both endogenous alleles encoding the IL-4 polypeptide. The CAR T cells having a reduced level of an IL-4 polypeptide can express a reduced level of the IL-4 polypeptide as compared to a comparable T cell lacking the disruption. The CAR T cells can have a reduced level of a TCF7 polypeptide. The CAR T cells having a reduced level of a TCF7 polypeptide can have a disruption in at least one endogenous allele encoding the TCF7 polypeptide. The CAR T cells having a reduced level of a TCF7 polypeptide can have a disruption in both endogenous alleles encoding the TCF7 polypeptide. The CAR T cells having a reduced level of a TCF7 polypeptide can express a reduced level of the TCF7 polypeptide as compared to a comparable T cell lacking the disruption. The CAR T cells can have a reduced level of a PTPN2 polypeptide. The CAR T cells having a reduced level of a PTPN2 polypeptide can have a disruption in at least one endogenous allele encoding the PTPN2 polypeptide. The CAR T cells having a reduced level of a PTPN2 polypeptide can have a disruption in both endogenous alleles encoding the PTPN2 polypeptide. The CAR T cells having a reduced level of a PTPN2 polypeptide can express a reduced level of the PTPN2 polypeptide as compared to a comparable T cell lacking the disruption. The CAR T cells can have a reduced level of a PTPN3 polypeptide. The CAR T cells having a reduced level of a PTPN3 polypeptide can have a disruption in at least one endogenous allele encoding the PTPN3 polypeptide. The CAR T cells having a reduced level of a PTPN3 polypeptide can have a disruption in both endogenous alleles encoding the PTPN3 polypeptide. The CAR T cells having a reduced level of a PTPN3 polypeptide can express a reduced level of the PTPN3 polypeptide as compared to a comparable T cell lacking the disruption.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. A schematic of an assay designed to induce exhaustion in CAR T cells. On Day 15, the CART19 cells were considered exhausted, assay was continued until Day 22 for more terminally exhausted CAR T-cells.

FIGS. 2A-2C. The exhaustion assay described in FIG. 1 resulted in CAR T cells with a reduced ability to proliferate (FIG. 2A), a reduced CD4⁺ T cell population (FIG. 2B), and a reduced ability to produce 3 or more cytokines at once (FIG. 2C). This shows evidence of CAR T Cell dysfunction. These figures represent data from three biological replicates. *p<0.05, **p<0.01, ***p<0.001, ****p<0.0001, one-way ANOVA.

FIGS. 3A-3F. The exhaustion assay described in FIG. 1 resulted in CAR T cells with increased expression of inhibitory receptors such as PD-1 (FIG. 3A), TIM-3 (FIG. 3B), CTLA-4 (FIG. 3C), and LAG-3 (FIG. 3D) on CAR T cells. The expression of the inhibitory receptors continues to increase as the assay continues from Day 15 to Day 22. Additionally, the exhaustion assay described in FIG. 1 resulted in CAR T cells that exhibited a reduced production of effector cytokines such as IL-2 (FIG. 3E) and TNF-α (FIG. 3F). This showed evidence of exhaustion-specific signs of CAR T-cell dysfunction. These figures represent data from three biological replicates. *p<0.05, **p<0.01, ***p<0.001, ****p<0.0001, one-way ANOVA.

FIGS. 4A-4B. CART19-28ζ cells from Day 8 and Day 22 of the exhaustion assay described in FIG. 1 were used to treat nod scid gamma (NSG) mice that were engrafted with a CD19⁺ JeKo-1 tumor. Tumor flux measurements indicated increased tumor burden when mice were treated with Day 22 exhausted CART19-28ζ cells as compared to treatment with Day 8 CART19-28ζ cells (FIG. 4A). There was also a decreased probability of survival in mice treated with the Day 22 CART19-28ζ cells as opposed to the Day 8 CART19-28ζ cells (FIG. 4B). Five mice were included in each group. Tumor flux data was analyzed with two-way ANOVA and overall survival data was analyzed with a Log-rank (Mantel-Cox) test. *p<0.05, **p<0.01, ***p<0.001, ****p<0.0001.

FIGS. 5A-5B. Using the in vitro exhaustion assay depicted in FIG. 1 with CAR T cells from three healthy donors, RNA sequencing was performed on Day 8 and Day 15. Comparing RNA sequencing of Day 8 to Day 15 samples revealed the development of a distinct transcriptomic profile (FIG. 5A). This transcriptomic profile was characterized by an upregulation of known exhaustion genes such as EOMES and IL10RA (FIG. 5B). These figures represent differential gene expression analysis (padj<0.05) from three biological replicates.

FIG. 6. Since ATAC sequencing was also performed on Days 8 and 15 of the in vitro exhaustion assays, the genes that were both differentially expressed and differentially accessible were identified by using the sequencing analysis pipeline depicted in FIG. 6.

FIGS. 7A-7B. After determining genes that were both differentially expressed and differentially accessible between Day 8 and Day 15 cells, Ingenuity Pathway Analysis software was used to determine the affected pathways and potential upstream regulators. Pathway analysis revealed an enrichment in the T cell exhaustion pathway (FIG. 7A). Additionally, potential upstream regulators include IL-2, Tcf-7, and IL-4. These figures represent results from differential gene expression analysis (padj<0.05) and differential gene accessibility analysis (padj<0.05) from three biological replicates.

FIGS. 8A-9B. In order to determine which transcription factors play a key role in the development of exhaustion, Taiji, a multi-omics data analysis framework was used. Performing Taiji analysis with the RNA and ATAC sequencing from Day 8 and Day 15 revealed an enrichment in several transcription factors in Day 15 samples, including EOMES (FIG. 8A). Additionally, Taiji analysis revealed a reduction in the AP-1 transcription factors FOSB and FOSL2 from Day 8 to Day 15 (FIG. 8A). This figure represents average gene rank values from three biological replicates in each condition. Further, mining of previously conducted ChIPseq experiments with ReMap revealed an increase in accessibility of binding sites for transcription factors such as RUNX3, BATF, and TBX21 in Day 15 samples (FIG. 8B). Differential peak analysis results originated from three biological replicates.

FIG. 9. To evaluate CAR T-cell dysfunction in a more clinically relevant setting, RNA and ATAC sequencing was performed on baseline Axi-cel products from 3 responders and 3 non-responders from the Zuma-1 clinical trial. After comparing genes that were both differentially expressed and differentially accessible when comparing responders to non-responders, only two genes, IL-4 and HLA-DQB1, were identified (FIG. 9). These genes were upregulated in baseline products from non-responders. Differential gene analysis was performed with six biological replicates in total, three biological replicates per condition. A cut-off of p-value <0.05 was used for differentially expressed genes.

FIGS. 10A-10B. Taiji analysis was used to investigate the differential role of transcription factors in baseline Axi-cel products from responders and non-responders. It was revealed that GATA3 and EOMES were enriched in patient non-responders (FIG. 10A). This figure represents average gene rank values from three biological replicates in each condition. Further, mining of previously conducted ChIPseq experiments with ReMap revealed an increase in accessibility of binding sites for transcription factors such as TBX21 and RunX3 in Day 15 samples (FIG. 10B). Differential peak analysis results originated from three biological replicates.

FIG. 11. After overlapping the differentially expressed genes from the in vitro exhaustion assay and the differentially expressed genes in the comparison of baseline responder and non-responder samples, only one gene was in found to be common, PTPN3.

FIGS. 12A-12B. Addition of PTPN3 cDNA to CART19 cells resulted in a decrease in cytotoxicity (FIG. 12A) and a decrease in proliferation (FIG. 12B). This is evidence of the induction of CAR T-cell dysfunction after PTPN3 polypeptide levels were increased. These figures represent data from one biological replicate with two technical replicates. **p<0.01, two-way ANOVA and a student's t-test, respectively.

FIG. 13. A schematic of a CRISPR screen designed to identify key genes that could be downregulated to prevent the development of exhaustion.

FIGS. 14A-14C. Overall CRISPR screen results (day 8 vs. day 22). Positive selection occurred in the CRISPR screen as evidenced by an increase in the gini index from Day 8 to Day 22 samples (FIG. 14A). PCA analysis also revealed clustering based on experimental condition as opposed to biological replicate (FIG. 14B). Volcano plot depicting positive and negative selection of genes. Two of the top positively selected genes include PTPN2 and CSF2 (FIG. 14C). Three biological replicates were used.

FIGS. 15A-15B. Gene Ontology enrichment analysis of the positively selected genes (p-value <0.001) when comparing Day 8 to Day 22 samples revealed as enrichment in pathways such as: the regulation of JAK-STAT cascade, negative regulation of IL-2 mediated signaling pathway and negative regulation of IL-4 mediated signaling pathway (FIG. 15A). Two out of three of the IL-4 targeting gRNAs resulted in a positive fold change after averaging the fold change from three biological replicates (FIG. 15B).

FIGS. 16A-16B. Absolute CD3 counts after 5-days of antigen-specific proliferation of CAR T-cells were treated with either 20 ng/mL human recombinant IL-4 (hrIL-4) or diluent (FIG. 16A). 48-hour cytotoxicity results of CART19-28ζ cells that were either treated with 20 ng/mL human recombinant IL-4 or treated with vehicle. CART19-28ζ cells were co-cultured with JeKo-1 cells that express luciferase. Cytotoxicity was measured by luminescence after luciferin was added to the co-culture. This figure represents data from three biological replicates. *p<0.05, **p<0.01, ***p<0.001, student's t-test and two-way ANOVA, respectively.

FIGS. 17A-17B. Continuous treatment of CART19-28ζ cells with 20 ng/mL hrIL-4 resulted in an increase in the expression of TIM-3 by Day 15 (FIG. 17A) and an increase in the transcription of EOMES by Day 15 (FIG. 17B). This figure represents data from three biological replicates. **p<0.01, student's t-test.

FIG. 18. Functional changes seen after the treatment of CART19-28ζ cells with 20 ng/mL hrIL-4 did not appear to be a result of an effect of hrIL-4 on JeKo-1 tumor cells. No significant changes in luminescence were seen when luciferase⁺ JeKo-1 cells were treated with 20 ng/mL hrIL-4. This figure represents data from six technical replicates. ns=not statistically significant, student's t-test.

FIGS. 19A-19C. The efficacy of a combination of CAR T cell therapy+IL-4 neutralization with a monoclonal antibody (mAb) was tested using a JeKo-1 xenograft mouse model in NSG mice. Combination of Day 8 CAR T Cells and 10 mg/kg IL-4 mAb resulted in reduced tumor burden (FIG. 19A), an increase in CAR T cell proliferation (FIG. 19B), and an increase in overall survival as compared to mice treated with Day 8 CAR T Cells (FIG. 19C). Five mice were included in each group. Tumor flux data was analyzed with two-way ANOVA, T cell proliferation was analyzed with a student's t-test, and overall survival data was analyzed with a Log-rank (Mantel-Cox) test. *p<0.05, **p<0.01, ***p<0.001, ****p<0.0001.

FIGS. 20A and 20B. There was enhanced chromatin accessibility at exhaustion-related gene loci (e.g., PDCD1 and ENTPD1) in the chronically stimulated samples (FIG. 20A) as well as enhanced motif accessibility for AP-1 and RUNX family members (FIG. 20B).

FIGS. 21A-21C. To further evaluate CAR T-cell dysfunction, RNA and ATAC sequencing was performed on baseline Axi-cel products from 6 responders and 6 non-responders from the Zuma-1 clinical trial. Interrogation of the transcriptome with RNA sequencing of pre-infusion axi-cel products from 6 responders and 6 non-responders showed clustering of non-responder samples (FIG. 21A). One of the top upregulated genes in CAR T cells from non-responders is IL-4 (FIG. 21B). Additionally, analysis of the differentially expressed genes with QIAGEN IPA identified IL-4 as one of the upstream regulators, along with other genes such as TNF and STAT3 (FIG. 21C) Differential gene analysis was performed with twelve biological replicates in total, six biological replicates per condition. A cut-off of p-value <0.05 was used for differentially expressed genes.

FIGS. 22A-22B. Analysis of chromatin changes between responder and non-responder samples revealed many similarities to the findings observed following chronic stimulation of healthy donor CART19-28ζ cells in the in vitro model for exhaustion. Motif analysis revealed an enrichment of motif binding sites for EOMES and PRDM1 (FIG. 22A). Additionally, non-responders showed similar enhancement of chromatin accessibility to exhausted cell populations at exhaustion-related gene loci such as PDCD1, HATVCR2 (TIM-3), and EOAES (FIG. 22B).

FIG. 23. Continuous treatment of CART19-28ζ cells with 20 ng/mL hrIL-4 resulted in an increase in the percent of CAR T cells expressing multiple inhibitory receptors by Day 15.

FIGS. 24A-24C. Continuous treatment of CAR T cells with hrIL-4 still enhanced the exhaustion profile by Day 15 when the CAR T cells were exposed to cell-free stimulation by CD19 beads. This is seen by a decrease in proliferative ability (FIG. 24A), a decrease in IL-2 production (FIG. 24B), and an increase in the percent of CAR T cells expressing multiple inhibitory receptors (FIG. 24C).

FIG. 25 contains graphs plotting the percent of IL-4 producing CD3 cells following IL-4 knockout in CART19-28ζ cells using gRNA1 or gRNA2 as compared to control gRNA. The percent of cells producing IL-4 was determined by intracellular staining for IL-4 by flow cytometry following four hours of antigen-specific CAR stimulation by co-culturing CART cells with JeKo-1 target cells at a 1:5 effector-to-target ratio. Three biological replicates; T-test; *=p<0.05. The DNA sequence encoding gRNA1 is TGATATCGCACTTGTGTCCG (SEQ ID NO:1), the DNA sequence encoding gRNA2 is CAAGTGCGATATCACCTTAC (SEQ ID NO:2), and the DNA sequence encoding a control gRNA is GTATTACTGATATTGGTGGG (SEQ ID NO:14).

FIG. 26 contains graphs plotting percent killing as measured with bioluminescent imaging after either IL-4 knockout using gRNA1 or gRNA2 as compared to control gRNA in CART19-28ζ cells co-cultured with luciferase+ Jeko-1 cells at various effector-to-target ratios for 24 hours. Three biological replicates; two-way ANOV; *=p<0.05. The DNA sequence encoding gRNA1 is TGATATCGCACTTGTGTCCG (SEQ ID NO:1), the DNA sequence encoding gRNA2 is CAAGTGCGATATCACCTTAC (SEQ ID NO:2), and the DNA sequence encoding a control gRNA is GTATTACTGATATTGGTGGG (SEQ ID NO:14).

FIGS. 27A-27G show the effect of IL-4 knockout on CAR T cell exhaustion. FIG. 27A) Absolute CD3⁺ cell count, as determined with flow cytometry, after chronically stimulated control gRNA CART19-28ζ cells or IL-4 knockout CART19-28ζ cells (using gRNA 1) were co-cultured with JeKo-1 target cells at a 1:1 ratio for 5 days (T-test with one biological replicate and three technical replicates). FIGS. 27B and 27C) The percent of IL-4 knockout CART19-28ζ cells (using gRNA 1) as compared with control gRNA CART19-28ζ cells producing the effector cytokines IL-2 and IFN-7, respectively. This was determined by strongly stimulating CAR T cells at a 1:5 effector to target cell ratio for four hours before performing intracellular staining for the cytokines (Paired T-test with three biological replicates, average of two technical replicates per biological replicate). FIG. 27D) Absolute CD3⁺ cell count, as determined with flow cytometry, after chronically stimulated control gRNA CAR T cells or IL-4 knockout CAR T cells (using gRNA 2) were co-cultured with JeKo-1 target cells at a 1:1 ratio for 5 days (T-test with one biological replicate and three technical replicates). FIGS. 27E and 27F) The percent of IL-4 knockout CART19-28ζ cells (using gRNA 2) as compared with control gRNA CART19-28ζ cells producing the effector cytokines IL-2 and IFN-γ, respectively. This was determined by strongly stimulating CAR T cells at a 1:5 effector to target cell ratio for four hours before performing intracellular staining for the cytokines (Paired T-test with two biological replicates, average of two technical replicates per biological replicate). FIG. 27G) Circle plots showing the percent of either control gRNA, IL-4 gRNA 1, or IL-4 gRNA 2 CART19-28ζ cells expressing 0, 1, 2, 3, or 4 inhibitory receptors following chronic stimulation with the in vitro model for exhaustion. This was determined with flow cytometric detection of CD3⁺ cells positive for PD-1, TIM-3, CTLA-4, and/or LAG-3 (One representative biological replicate out of three tested). (*p<0.05, **p<0.01). The DNA sequence encoding gRNA1 is TGATATCGCACTTGTGTCCG (SEQ ID NO:1), the DNA sequence encoding gRNA2 is CAAGTGCGATATCACCTTAC (SEQ ID NO:2), and the DNA sequence encoding a control gRNA is GTATTACTGATATTGGTGGG (SEQ ID NO:14).

FIGS. 28A-28G show the effect of human recombinant IL-4 on the activity of CART19-BBC cells. FIG. 28A) Percent killing as measured with bioluminescent imaging after CART19-BBζ cells were co-cultured with luciferase⁺ JeKo-1 cells at various E:T cell ratios for 48 hours in the presence of either 20 ng/mL human recombinant IL-4 (hrIL-4) or diluent control (Two-way ANOVA with three biological replicates, two technical replicates per biological replicate). FIG. 28B) Absolute CD3⁺ cell count as measured with flow cytometry after CART19-BBζ cells were co-cultured with JeKo-1 cells at a 1:1 E:T cell ratio for five days in the presence of either 20 ng/mL hrIL-4 or diluent control (Paired t-test with three biological replicates, two technical replicates per biological replicate). FIG. 28C) Absolute CD3⁺ cell count as measured with flow cytometry after CART19-BBζ cells that had been chronically stimulated either in the presence of 20 ng/mL hrIL-4 or diluent for one week were co-cultured with JeKo-1 cells at a 1:1 E:T cell ratio for five days (Paired t-test with three biological replicates, two technical replicates per biological replicate). FIG. 28D) The percent of CD107a+ T cells in CART19-BBζ cells that have been chronically stimulated for a week in the presence of 20 ng/mL hrIL-4 or diluent. This is determined by flow cytometry after strongly stimulating the chronically stimulated CAR T cells with CD19⁺ target cells at a 1:5 effector-to-target cell ratio for four hours (Paired T-test with three biological replicates, average of two technical replicates). FIGS. 28E and 28F) The percent of CART19-BBQ cells producing the effector cytokines IFN-γ and IL-2, respectively following one week of chronic stimulation in the presence of 20 ng/mL hrIL-4 or diluent. This was determined by strongly stimulating chronically stimulated CAR T cells at a 1:5 effector to target cell ratio for four hours before performing intracellular staining for the cytokines (Paired T-test with two biological replicates, average of two technical replicates per biological replicate). FIG. 28G) The percent of CART19-BBζ cells expressing three or more inhibitory receptors 5 following chronic stimulation in the presence of either 20 ng/mL hrIL-4 or diluent. This was determined with flow cytometric detection of CD3⁺ cells positive for PD-1, TIM-3, CTLA-4, and/or LAG-3 (Paired T-test with three biological replicates, average of two technical replicates). (*p<0.05, **p<0.01, ***p<0.001, ****p<0.0001).

### DETAILED DESCRIPTION

This document provides methods and materials for generating T cells (e.g., CAR T cells) having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells). In some cases, a T cell (e.g., a CAR T cell) can be engineered to KO a nucleic acid encoding an IL-4 polypeptide to reduce IL-4 polypeptide expression in that T cell (e.g., as compared to a comparable T cell that is not engineered to KO a nucleic acid encoding an IL-4 polypeptide). AT cell that is engineered to KO a nucleic acid encoding an IL-4 polypeptide can also be referred to herein as an IL-4 KO T cell, an IL-4^{k/o} T cell, or an IL-4^{KO} T cell. In some cases, a T cell (e.g., a CAR T cell) can be engineered to KO a nucleic acid encoding a TCF7 polypeptide to reduce TCF7 polypeptide expression in that T cell (e.g., as compared to a comparable T cell that is not engineered to KO a nucleic acid encoding a TCF7 polypeptide). AT cell that is engineered to KO a nucleic acid encoding a TCF7 polypeptide can also be referred to herein as a TCF7 KO T cell, a TCF7^{k/o} T cell, or a TCF7^{KO} T cell. In some cases, a T cell (e.g., a CAR T cell) can be engineered to KO a nucleic acid encoding a PTPN2 polypeptide to reduce PTPN3 polypeptide expression in that T cell (e.g., as compared to a comparable T cell that is not engineered to KO a nucleic acid encoding a PTPN2 polypeptide). AT cell that is engineered to KO a nucleic acid encoding a PTPN2 polypeptide can also be referred to herein as a PTPN2 KO T cell, a PTPN2^{k/o} T cell, or a PTPN2^{KO} T cell. In some cases, a T cell (e.g., a CAR T cell) can be engineered to KO a nucleic acid encoding a PTPN3 polypeptide to reduce PTPN3 polypeptide expression in that T cell (e.g., as compared to a comparable T cell that is not engineered to KO a nucleic acid encoding a PTPN3 polypeptide). AT cell that is engineered to KO a nucleic acid encoding a PTPN3 polypeptide can also be referred to herein as a PTPN3 KO T cell, a PTPN3^{k/o} T cell, or a PTPN3^{KO} T cell.

The term “reduced level” as used herein with respect to a level of an IL-4 polypeptide, a TCF7 polypeptide, a PTPN2 polypeptide, or a PTPN3 polypeptide refers to any level that is lower than a reference level of that polypeptide. The term “reference level” as used herein with respect to an IL-4 polypeptide, a TCF7 polypeptide, a PTPN2 polypeptide, or a PTPN3 polypeptide refers to the level of that polypeptide typically observed in control T cells from one or more mammals (e.g., humans) not engineered to have a reduced level of that polypeptide as described herein. Control T cells can include, without limitation, T cells that are wild-type T cells. In some cases, a reduced level of an IL-4 polypeptide, a TCF7 polypeptide, a PTPN2 polypeptide, or a PTPN3 polypeptide can be an undetectable level of that polypeptide. In some cases, a reduced level of an IL-4 polypeptide, a TCF7 polypeptide, a PTPN2 polypeptide, and/or a PTPN3 polypeptide can be an eliminated level of that polypeptide.

In some cases, a T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide is less likely to undergo exhaustion (e.g., as compared to a CAR T cell that is not engineered to have a reduced level of an IL-4 polypeptide, a reduced level of a TCF7 polypeptide, a reduced level of a PTPN2 polypeptide, and/or a reduced level of a PTPN3 polypeptide). For example, reducing a level of an IL-4 polypeptide, reducing a level of a TCF7 polypeptide, a reduced level of a PTPN2 polypeptide, and/or reducing a level of a PTPN3 polypeptide in a T cell can be effective to delay the onset of exhaustion in the T cell by, for example, 10, 20, 30, 40, 50, 60, 70, 80, 90, 95, or more percent. For example, a T cell having a reduced level of an IL-4 polypeptide, a reduced level of a TCF7 polypeptide, a reduced level of a PTPN2 polypeptide, and/or a reduced level of a PTPN3 polypeptide can maintain one or more T cell functions for at least 1 week. In some cases, a T cell having a reduced level of an IL-4 polypeptide, a reduced level of a TCF7 polypeptide, a reduced level of a PTPN2 polypeptide, and/or a reduced level of a PTPN3 polypeptide can maintain one or more T cell functions for from about 1 week to about 2 years (e.g., from about 1 week to about 1.5 years, from about 1 week to about 1 year, from about 1 week to about 9 months, from about 1 week to about 6 months, from about 1 week to about 4 months, from about 1 week to about 3 months, from about 1 week to about 2 months, from about 1 week to about 1 month, from about 1 week to about 4 weeks, from about 1 week to about 3 weeks, from about 1 week to about 2 weeks, from about 2 weeks to about 2 years, from about 3 weeks to about 2 years, from about 4 weeks to about 2 years, from about 1 month to about 2 years, from about 2 months to about 2 years, from about 3 months to about 2 years, from about 4 months to about 2 years, from about 6 months to about 2 years, from about 9 months to about 2 years, from about 1 year to about 2 years, from about 1.5 years to about 2 years, from about 2 weeks to about 1.5 years, from about 3 weeks to about 1 year, from about 1 month to about 9 months, from about 2 months to about 6 months, from about 3 months to about 4 months, from about 2 weeks to about 4 weeks, from about 1 month to about 4 months, from about 2 months to about 6 months, from about 3 months to about 9 months, or from about 6 months to about 1.5 years).

Any appropriate method can be used to determine whether or not one or more T cells (e.g., T cells having a reduced level of an IL-4 polypeptide, a reduced level of a TCF7 polypeptide, a reduced level of a PTPN2 polypeptide, and/or a reduced level of a PTPN3 polypeptide) are exhausted. Examples of methods that can be used to evaluate T cell (e.g., CAR T cell) exhaustion include, without limitation, testing the T cell's ability to proliferation, to maintain a CD4⁺ population, to produce multiple cytokines at once, to produce effector cytokines such as IL-2 and TNF-α, and to express inhibitory receptors.

In some cases, a T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide can have enhanced CAR T cell function such as improved antitumor activity, improved proliferation, improved cell killing (e.g., improved killing of tumor cells), improved CD4⁺ T cell levels, improved persistence, and improved ability to produce several cytokines at once (e.g., as compared to a CAR T cell that is not engineered to have a reduced level of an IL-4 polypeptide, a reduced level of a TCF7 polypeptide, a reduced level of a PTPN2 polypeptide, and/or a reduced level of a PTPN3 polypeptide). Any appropriate method can be used to assess one or more functions of T cells (e.g., T cells having a reduced level of an IL-4 polypeptide, a reduced level of a TCF7 polypeptide, a reduced level of a PTPN2 polypeptide, and/or a reduced level of a PTPN3 polypeptide). Examples of methods that can be used to evaluate T cell (e.g., CAR T cell) functions include, without limitation, the treatment of in vivo tumor models, proliferation assays (to determine absolute T cell numbers following antigen-specific stimulation), cytotoxicity assays (e.g., to evaluate whether or not T cells (e.g., CAR T cells) are effective at killing target cells, cell number determinations, proliferation assays, determining CD4⁺ cell populations at different timepoints, and degranulation assays.

A T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide can be any appropriate T cell. A T cell can be a naïve T cell. Examples of T cells that can be engineered to have a reduced level of an IL-4 polypeptide, a reduced level of a TCF7 polypeptide, a reduced level of a PTPN2 polypeptide, and/or a reduced level of a PTPN3 polypeptide as described herein include, without limitation, cytotoxic T cells, helper T cells, CD4⁺ T cells, and CD8⁺ T cells. For example, a T cell that can be engineered to have a reduced level of an IL-4 polypeptide, a reduced level of a TCF7 polypeptide, a reduced level of a PTPN2 polypeptide, and/or a reduced level of a PTPN3 polypeptide as described herein can be a CAR T cell. In some cases, one or more T cells designed to have a reduced level of an IL-4 polypeptide, a reduced level of a TCF7 polypeptide, a reduced level of a PTPN2 polypeptide, and/or a reduced level of a PTPN3 polypeptide can be T cells that were obtained from a mammal (e.g., a mammal having cancer) that is to be treated with those T cells designed to have a reduced level of an IL-4 polypeptide, a reduced level of a TCF7 polypeptide, a reduced level of a PTPN2 polypeptide, and/or a reduced level of a PTPN3 polypeptide. For example, T cells can be obtained from a mammal to be treated with the materials and method described herein.

A T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide can be generated using any appropriate method. In some cases, a T cell (e.g., a CAR T cell) can be treated with one or more inhibitors to reduce polypeptide expression in that T cell (e.g., as compared to a T cell that was not treated with the one or more inhibitors). For example, a T cell (e.g., a CAR T cell) can be treated with one or more IL-4 polypeptide inhibitors to reduce a level of IL-4 polypeptides in that T cell (e.g., as compared to a T cell that was not treated with the one or more IL-4 polypeptide inhibitors). For example, a T cell (e.g., a CAR T cell) can be treated with one or more TCF7 polypeptide inhibitors to reduce a level of TCF7 polypeptide in that T cell (e.g., as compared to a T cell that was not treated with the one or more TCF7 polypeptide inhibitors). For example, a T cell (e.g., a CAR T cell) can be treated with one or more PTPN2 polypeptide inhibitors to reduce a level of PTPN2 polypeptides in that T cell (e.g., as compared to a T cell that was not treated with the one or more PTPN2 polypeptide inhibitors). For example, a T cell (e.g., a CAR T cell) can be treated with one or more PTPN3 polypeptide inhibitors to reduce a level of PTPN3 polypeptides in that T cell (e.g., as compared to a T cell that was not treated with the one or more PTPN3 polypeptide inhibitors).

In some cases, T cells (e.g., CAR T cells) and one or more inhibitors can be administered to a mammal (e.g., a human) having cancer to reduce polypeptide expression within the mammal (e.g., to generate CAR T cells having a reduced susceptibility to T cell exhaustion within the mammal). For example, T cells (e.g., CAR T cells) and one or more IL-4 polypeptide inhibitors can be administered to a mammal (e.g., a human) having cancer (e.g., to generate CAR T cells having a reduced susceptibility to T cell exhaustion within the mammal). For example, a T cell (e.g., a CAR T cell) and one or more TCF7 polypeptide inhibitors can be administered to a mammal (e.g., a human) having cancer (e.g., to generate CAR T cells having a reduced susceptibility to T cell exhaustion within the mammal). For example, a T cell (e.g., a CAR T cell) and one or more PTPN2 polypeptide inhibitors can be administered to a mammal (e.g., a human) having cancer (e.g., to generate CAR T cells having a reduced susceptibility to T cell exhaustion within the mammal). For example, a T cell (e.g., a CAR T cell) and one or more PTPN3 polypeptide inhibitors can be administered to a mammal (e.g., a human) having cancer (e.g., to generate CAR T cells having a reduced susceptibility to T cell exhaustion within the mammal). In some cases, T cells (e.g., CAR T cells) having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) and one or more inhibitors can be administered to a mammal (e.g., a human) having cancer to reduce polypeptide expression within the mammal (e.g., to generate CAR T cells having a reduced susceptibility to T cell exhaustion within the mammal).

An IL-4 polypeptide inhibitor can be any appropriate IL-4 polypeptide inhibitor. An IL-4 polypeptide inhibitor can be an inhibitor of IL-4 polypeptide expression or an inhibitor of IL-4 polypeptide activity. In some cases, an IL-4 inhibitor can inhibit one or more polypeptides that can regulate production of an IL-4 polypeptide (e.g., one or more polypeptides that are upstream regulators of an IL-4 polypeptide). Examples of compounds that can inhibit IL-4 polypeptide activity include, without limitation, antibodies (e.g., neutralizing antibodies) that target (e.g., target and bind) to an IL-4 polypeptide, and small molecules that target (e.g., target and bind) to an IL-4 polypeptide. Examples of compounds that can inhibit of IL-4 polypeptide expression include, without limitation, nucleic acid molecules designed to induce RNA interference of polypeptide expression of an IL-4 polypeptide (e.g., a siRNA molecule or a shRNA molecule), antisense molecules, and miRNAs. In some cases, an IL-4 polypeptide inhibitor that can be contacted with a T cell (e.g., CAR T cell) to reduce a level of an IL-4 polypeptide within the T cell can be as described elsewhere (see, e.g., Quinnell et al., ACS Chem. Biol., 15, 10, 2649-2654 (2020)).

A TCF7 polypeptide inhibitor can be any appropriate TCF7 polypeptide inhibitor. A TCF7 polypeptide inhibitor can be an inhibitor of TCF7 polypeptide expression or an inhibitor of TCF7 polypeptide activity. Examples of compounds that can inhibit TCF7 polypeptide activity include, without limitation, antibodies (e.g., neutralizing antibodies) that target (e.g., target and bind) to a TCF7 polypeptide, and small molecules that target (e.g., target and bind) to a TCF7 polypeptide. Examples of compounds that can inhibit of TCF7 polypeptide expression include, without limitation, nucleic acid molecules designed to induce RNA interference of polypeptide expression of a TCF7 polypeptide (e.g., a siRNA molecule or a shRNA molecule), antisense molecules, and miRNAs.

A PTPN2 polypeptide inhibitor can be any appropriate PTPN2 polypeptide inhibitor. A PTPN2 polypeptide inhibitor can be an inhibitor of PTPN2 polypeptide expression or an inhibitor of PTPN2 polypeptide activity. Examples of compounds that can inhibit PTPN2 polypeptide activity include, without limitation, antibodies (e.g., neutralizing antibodies) that target (e.g., target and bind) to a PTPN2 polypeptide, and small molecules that target (e.g., target and bind) to a PTPN2 polypeptide. Examples of compounds that can inhibit of PTPN2 polypeptide expression include, without limitation, nucleic acid molecules designed to induce RNA interference of polypeptide expression of a PTPN2 polypeptide (e.g., a siRNA molecule or a shRNA molecule), antisense molecules, and miRNAs. Examples of PTPN2 polypeptide inhibitors that can be contacted with a T cell (e.g., CAR T cell) to reduce a level of a PTPN2 polypeptide within the T cell include, without limitation, ABBV-CLS-484 and ABBV-CLS-579.

A PTPN3 polypeptide inhibitor can be any appropriate PTPN3 polypeptide inhibitor. A PTPN3 polypeptide inhibitor can be an inhibitor of PTPN3 polypeptide expression or an inhibitor of PTPN3 polypeptide activity. Examples of compounds that can inhibit PTPN3 polypeptide activity include, without limitation, antibodies (e.g., neutralizing antibodies) that target (e.g., target and bind) to a PTPN3 polypeptide, and small molecules that target (e.g., target and bind) to a PTPN3 polypeptide. Examples of compounds that can inhibit of PTPN3 polypeptide expression include, without limitation, nucleic acid molecules designed to induce RNA interference of polypeptide expression of a PTPN3 polypeptide (e.g., a siRNA molecule or a shRNA molecule), antisense molecules, and miRNAs.

In some cases, a T cell (e.g., a CAR T cell) can be engineered to KO a nucleic acid encoding an IL-4 polypeptide to reduce IL-4 polypeptide expression in that T cell, can be engineered to KO a nucleic acid encoding a TCF7 polypeptide to reduce TCF7 polypeptide expression in that T cell, can be engineered to KO a nucleic acid encoding a PTPN2 polypeptide to reduce PTPN2 polypeptide expression in that T cell, and/or can be engineered to KO a nucleic acid encoding a PTPN3 polypeptide to reduce PTPN3 polypeptide expression in that T cell.

In some cases, a T cell (e.g., a CAR T cell) can be engineered to KO a nucleic acid encoding an IL-4 polypeptide to reduce IL-4 polypeptide expression in that T cell. For example, at least one endogenous allele of a nucleic acid encoding an IL-4 polypeptide can be disrupted (e.g., knocked out) to generate a T cell (e.g., a CAR T cell) having a reduced level of an IL-4 polypeptide. For example, both endogenous alleles of a nucleic acid encoding an IL-4 polypeptide can be disrupted (e.g., knocked out) to generate a T cell (e.g., a CAR T cell) having a reduced level of an IL-4 polypeptide.

In some cases, a T cell (e.g., a CAR T cell) can be engineered to KO a nucleic acid encoding a TCF7 polypeptide to reduce TCF7 polypeptide expression in that T cell. For example, at least one endogenous allele of a nucleic acid encoding a TCF7 polypeptide can be disrupted (e.g., knocked out) to generate a T cell (e.g., a CAR T cell) having a reduced level of a TCF7 polypeptide. For example, both endogenous alleles of a nucleic acid encoding a TCF7 polypeptide can be disrupted (e.g., knocked out) to generate a T cell (e.g., a CAR T cell) having a reduced level of a TCF7 polypeptide.

In some cases, a T cell (e.g., a CAR T cell) can be engineered to KO a nucleic acid encoding a PTPN2 polypeptide to reduce PTPN2 polypeptide expression in that T cell. For example, at least one endogenous allele of a nucleic acid encoding a PTPN2 polypeptide can be disrupted (e.g., knocked out) to generate a T cell (e.g., a CAR T cell) having a reduced level of a PTPN2 polypeptide. For example, both endogenous alleles of a nucleic acid encoding a PTPN2 polypeptide can be disrupted (e.g., knocked out) to generate a T cell (e.g., a CAR T cell) having a reduced level of a PTPN2 polypeptide.

In some cases, a T cell (e.g., a CAR T cell) can be engineered to KO a nucleic acid encoding a PTPN3 polypeptide to reduce PTPN3 polypeptide expression in that T cell. For example, at least one endogenous allele of a nucleic acid encoding a PTPN3 polypeptide can be disrupted (e.g., knocked out) to generate a T cell (e.g., a CAR T cell) having a reduced level of a PTPN3 polypeptide. For example, both endogenous alleles of a nucleic acid encoding a PTPN3 polypeptide can be disrupted (e.g., knocked out) to generate a T cell (e.g., a CAR T cell) having a reduced level of a PTPN3 polypeptide.

In some cases, when a T cell (e.g., a CAR T cell) is engineered to KO a nucleic acid encoding an IL-4 polypeptide to reduce IL-4 polypeptide expression in that T cell, is engineered to KO a nucleic acid encoding a TCF7 polypeptide to reduce TCF7 polypeptide expression in that T cell, is engineered to KO a nucleic acid encoding a PTPN2 polypeptide to reduce PTPN2 polypeptide expression in that T cell, and/or is engineered to KO a nucleic acid encoding a PTPN3 polypeptide to reduce PTPN3 polypeptide expression in that T cell, any appropriate method can be used to KO a nucleic acid. Examples of techniques that can be used to knock out a nucleic acid encoding an IL-4 polypeptide, a nucleic acid encoding a TCF7 polypeptide, a nucleic acid encoding a PTPN2 polypeptide, and/or a nucleic acid encoding a PTPN3 polypeptide include, without limitation, gene editing, homologous recombination, non-homologous end joining, microhomology end joining, and base editing For example, gene editing (e.g., with engineered nucleases) can be used to knock out a nucleic acid encoding an IL-4 polypeptide, a nucleic acid encoding a TCF7 polypeptide, a nucleic acid encoding a PTPN2 polypeptide, and/or a nucleic acid encoding a PTPN3 polypeptide. Nucleases useful for genome editing include, without limitation, CRISPR-associated (Cas) nucleases, zinc finger nucleases (ZFNs), transcription activator-like effector (TALE) nucleases, and homing endonucleases (HE; also referred to as meganucleases).

In some cases, a clustered regularly interspaced short palindromic repeat (CRISPR)/Cas system can be used (e.g., can be introduced into one or more T cells) to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide. A CRISPR/Cas system used to KO a nucleic acid can include a guide RNA (gRNA) that is complementary to the target nucleic acid (e.g., a nucleic acid encoding an IL-4 polypeptide, a nucleic acid encoding a TCF7 polypeptide, a nucleic acid encoding a PTPN2 polypeptide, and/or a nucleic acid encoding a PTPN3 polypeptide). Examples of nucleic acids that can encode gRNAs that are specific to a nucleic acid encoding an IL-4 polypeptide include, without limitation, TGATATCGCACTTGTGTCCG (SEQ ID NO:1), CAAGTGCGATATCACCTTAC (SEQ ID NO:2), and GGTTCCTGTCGAGCCGTTTC (SEQ ID NO:3). In some cases, a gRNA can be designed based on a sequence of a nucleic acid encoding an IL-4 polypeptide. Examples of nucleic acids encoding an IL-4 polypeptide sequence include, without limitation, those set forth in National Center for Biotechnology Information (NCBI) accession no. NM_000589, accession no. NM_001354990.2, and accession no. NM_172348.3. For example, a gRNA specific to a nucleic acid encoding an IL-4 polypeptide can be as described elsewhere (see, e.g., Sanjana et al., Nat. Methods, 11(8):783-4 (2014)).

Examples of nucleic acids that can encode gRNAs that are specific to a nucleic acid encoding a TCF7 polypeptide include, without limitation, GTCGCTCGTGAACGAGTCCG (SEQ ID NO:4), CGCGCTGTCGCGAGAAGAGC (SEQ ID NO:5), and CACGAGCGACGACTTGAGCT (SEQ ID NO:6). In some cases, a gRNA can be designed based on a sequence of a nucleic acid encoding a TCF7 polypeptide. Examples of nucleic acids encoding a TCF7 polypeptide sequence include, without limitation, those set forth in NCBI accession no. NM_213648.5, accession no. NM_003202.5, and accession no. NM_001134851.4. For example, a gRNA specific to a nucleic acid encoding a TCF7 polypeptide can be as described elsewhere (see, e.g., Rutishauser et al., JCI insight, 6(3):(2021); and Zhou et al., Science Immunology, 7(71):eabm1920 (2022)).

Examples of nucleic acids that can encode gRNAs that are specific to a nucleic acid encoding a PTPN2 polypeptide include, without limitation, CTCTTCGAACTCCCGCTCGA (SEQ ID NO:7), AGTTGGATACTCAGCGTCGC (SEQ ID NO:8), and CCATGACTATCCTCATAGAG (SEQ ID NO:9). In some cases, a gRNA can be designed based on a sequence of a nucleic acid encoding a PTPN2 polypeptide. Examples of nucleic acids encoding a PTPN2 polypeptide sequence include, without limitation, those set forth in NCBI accession no. NM_002828.4, accession no. NM_080422.3, and accession no. NM_080423.3. For example, a gRNA specific to a nucleic acid encoding a PTPN2 polypeptide can be as described elsewhere (see, e.g., Zhang et al., J. Periodontal Research, 53(3):467-477 (2018); and LaFleur et al., Nature Immunology, 20(10):1335-1347 (2019)).

Examples of nucleic acids that can encode gRNAs that are specific to a nucleic acid encoding a PTPN3 polypeptide include, without limitation, ACAGCATGATGACGACTCCG (SEQ ID NO: 10), GGATATGGTGCACAACCACC (SEQ ID NO: 11), and CTGAAGATGGATATTTGCGA (SEQ ID NO:12). In some cases, a gRNA can be designed based on a sequence of a nucleic acid encoding a PTPN3 polypeptide. Examples of nucleic acids encoding a PTPN3 polypeptide sequence include, without limitation, those set forth in NCBI accession no. NM_002829.4, accession no. NM_001145368.2, and accession no. NM_001145369.2. For example, a gRNA specific to a nucleic acid encoding a PTPN3 polypeptide can be as described elsewhere (see, e.g., Sanjana et al, Nat. Methods, August; 11(4783-4 (2014)).

A CRISPR/Cas system used to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can include any appropriate Cas nuclease. Examples of Cas nucleases include, without limitation, Cas1, Cas2, Cas3, Cas9, Cas10, Cpf1, Cas12a, and ErCas12a. In some cases, a Cas component of a CRISPR/Cas system designed to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can be a Cas9 nuclease. An exemplary Cas9 nuclease can have the amino acid sequence set forth in Example 7. For example, the Cas9 nuclease of a CRISPR/Cas9 system described herein can be a Streptococcus pyogenes Cas9 nuclease (see, e.g., Cox et al., Leukemia, 36(6):1635-1645 (2022)).

Components of a CRISPR/Cas system (e.g., a gRNA and a Cas nuclease) used to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can be introduced into one or more T cells (e.g., CAR T cells) in any appropriate format. In some cases, a component of a CRISPR/Cas system can be introduced into one or more T cells as a nucleic acid encoding a gRNA and/or a nucleic acid encoding a Cas nuclease. For example, a nucleic acid encoding at least one gRNA and a nucleic acid encoding at least one Cas nuclease (e.g., a Cas9 nuclease) can be introduced into one or more T cells. In some cases, a component of a CRISPR/Cas system can be introduced into one or more T cells as a gRNA and/or as a Cas nuclease. For example, at least one gRNA and at least one Cas nuclease (e.g., a Cas9 nuclease) can be introduced into one or more T cells.

In some cases, a ZFN system can be used can be used (e.g., can be introduced into one or more T cells) to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide. A ZFN system used to KO a nucleic acid can include a polypeptide including (a) a DNA-binding domain (e.g., zinc fingers) that that is complementary to a target nucleic acid (e.g., a nucleic acid encoding an IL-4 polypeptide, a nucleic acid encoding a TCF7 polypeptide, a nucleic acid encoding a PTPN2 polypeptide, and/or a nucleic acid encoding a PTPN3 polypeptide), and (b) a nuclease domain (e.g., a nuclease domain that can created double-strand breaks). A ZFN system used to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can include any appropriate nuclease domain. In some cases, a nuclease domain of a ZFN system designed to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can be a Fok1 nuclease domain.

In some cases, a TALEN system can be used can be used (e.g., can be introduced into one or more T cells) to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide. A TALEN system used to KO a nucleic acid can include a polypeptide including (a) a transcription activator-like (TAL) effector DNA-binding domain directing the nuclease to a target nucleic acid (e.g., a nucleic acid encoding an IL-4 polypeptide, a nucleic acid encoding a TCF7 polypeptide, a nucleic acid encoding a PTPN2 polypeptide, and/or a nucleic acid encoding a PTPN3 polypeptide), and (b) a nuclease domain (e.g., a nuclease domain that can created double-strand breaks). A TALEN system used to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can include any appropriate nuclease. In some cases, a nuclease can be a non-specific nuclease. In some cases, a nuclease can function as a dimer. In some cases, a nuclease of a TALEN system designed to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can be a Fok1 nuclease.

Components of a gene-editing system (e.g., a CRISPR/Cas system) used to KO a nucleic acid encoding an IL-4 polypeptide, to KO a nucleic acid encoding a TCF7 polypeptide, to KO a nucleic acid encoding a PTPN2 polypeptide, and/or to KO a nucleic acid encoding a PTPN3 polypeptide can be introduced into one or more T cells (e.g., CAR T cells) using any appropriate method. A method of introducing components of a gene-editing system into a T cell can be a physical method. A method of introducing components of a gene-editing system into a T cell can be a chemical method. A method of introducing components of a gene-editing system into a T cell can be a particle-based method. Examples of methods that can be used to introduce components of a gene-editing system into one or more T cells include, without limitation, electroporation, transfection (e.g., lipofection), transduction (e.g., viral vector mediated transduction), microinjection, nucleofection, Cell Squeeze®, and microfluidics.

A T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, a T cell can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor expressed in a T cell having a reduced level of an IL-4 polypeptide, a reduced level of a TCF7 polypeptide, a reduced level of a PTPN2 polypeptide, and/or a reduced level of a PTPN3 polypeptide as described herein include, without limitation, cluster of differentiation 19 (CD19), mucin 1 (MUC-1), human epidermal growth factor receptor 2 (HER-2), estrogen receptor (ER), epidermal growth factor receptor (EGFR), alphafetoprotein (AFP), carcinoembryonic antigen (CEA), CA-125, epithelial tumor antigen (ETA), melanoma-associated antigen (MAGE), CD33, CD123, CLL-1, E-Cadherin, folate receptor alpha, folate receptor beta, IL13R, EGFRviii, CD22, CD20, kappa light chain, lambda light chain, desmopressin, CD44v, CD45, CD30, CD5, CD7, CD2, CD38, BCMA, CD138, FAP, CS-1, C-met, TSHR, and EphA3. For example, a T cell having a reduced level of an IL-4 polypeptide, a reduced level of a TCF7 polypeptide, a reduced level of a PTPN2 polypeptide, and/or a reduced level of a PTPN3 polypeptide can be designed to express an antigen receptor targeting CD19.

In some cases, a CAR can be designed to include a single chain antibody (e.g., a scFv) targeting a tumor antigen. For example, a CAR can be designed to include a single chain antibody as set forth in Table 1.

TABLE 1
Exemplary CARs for targeting tumor antigens.

| Antigen | scFv antibody name | Reference(s) |
|---|---|---|
| CD19 | FMC63 | Milone et al., Mol. Ther., 17(8): 1453-64 (2009) (PMID: 26330164) |
| | MOR208 | Kellner et al., Leukemia, 27(7): 1595-8 (2013) (PMID: 23277329) |
| | Humanized scFv | International Patent Application Publication No. WO2015157252 |
| | 4G7 | EP Patent No. EP2997141 |
| | Low affinity scFv | Chinese Patent No. CN107406517 |
| | FMC63 CAR | Kang et al., Int. J. Mol. Sci., 21(23): 9163 (2020) |
| | KHYG-1 and 4G7 | |
| | CAR KHYG-1 | |
| MUC-1 | 5E5 | Posey et al., Immunity, 45(5): 947-948 (2016) (PMID: 27851918) |
| HER-2 | 4D5 | Ohnishi et al., Br. J. Cancer, 71(5): 969-73 (1995) (PMID: 7734322) |
| | | Forsberg et al., Cancer Res., 79(5): 899-904 (2019) (PMID: 30622115) |
| | | Nellan et al., J. Immunother. Cancer, 6(1): 30 (2018) (PMID: 29712574) |
| | | Priceman et al., Clin Cancer Res., 24(1): 95-105 (2018) (PMID: |
| | | 29061641) |
| | FRP5 | Bielamowics et al., Neuro. Oncol., 20(4): 506-518 (2018) (PMID: |
| | | 29016929) |
| | K70H-28z, K70L- | Zhou et al., Front. Cell Develop. Biol., 10: (2022). |
| | 28z, KSAbH-28z and | |
| | KSAbL-28z | |
| EGFR | M27 | Jiang et al., Cancer Immunol. Res., 6(11): 1314-1326 (2018) (PMID: |
| | | 30201736) |
| | Cetuximab | Caruso et al., Cancer Res., 75(17): 3505-18 (2015) (PMID: 26330164) |
| Folate receptor alpha | C4 based | Ao et al., J. Immunother., 42(8): 284-296 (2019) (PMID: 31261167) |
| | MOv19 | Song et al., J. Hematol. Oncol., 9(1): 56 (2016) (PMID: 27439908) |
| Mesothelin | SS1 | Haas et al., Mol. Ther., S1525-0016(19)30328-4 (2019) (PMID: |
| | | 31420241) |
| | M clone | Adusumilli et al., Sci. Transl. Med., 6(261): 261ra151 (2014) (PMID: |
| | | 25378643) |
| AFP | ET1402L1 | Liu et al., Clin. Cancer Res., 23(2): 478-488 (2017) (PMID: 27535982) |
| CEA | Anti-CEA scFv | Chi et al., Cancer Med., 8(10): 4753-4765 (2019) (PMID: 31237116) |
| | CEACAM5 | Thistlethwaite et al., Cancer Immunol. Immunother., 66(11): 1425-1436 |
| | | (2017) (PMID: 28660319) |
| | hMN14 | Katz et al., Clin. Cancer Res., 21(14): 3149-59 (2015) (PMID: |
| | | 25850950) |
| CD123 | 22172, 22176 | Gill et al., Blood, 123(15): 2343-54 (2014) (PMID: 24596416) |
| | Humanized scFv | US Patent Application Publication No. 2016/0068601 |
| | Humanized scFv | EP Patent No. EP2968415 |
| | Tagrazofusp based | Pemmaraju et al. N. Engl. J. Med., 380(17): 1628-1637 (2019) (PMID: |
| | | 31018069) |
| CD33 | MY96 | Kenderian et al., Leukemia, 29(8): 1637-47 (2015) (PMID: 25721896) |
| | Humanized scFv | International Patent Application Publication No. WO2016014576 |
| CLEC12A | Humanized scFv | US Patent Application Publication No. US20160051651 |
| | CLL1 scFv | International Patent Application Publication No. WO2016120219 |
| CD22 | M0971 | Fry et al., Nat. Med., 24(1): 20-28 (2018) (PMID: 29155426) |
| | Humanized scFv | International Patent Application Publication No. WO/2016/164731 |
| | clones | |
| | Inotuzumab based | Kantarjian et al., N. Engl. J. Med., 375(8): 740-53 (2016) (PMID: |
| | | 27292104) |
| | Moxetumomab based | Kreitman et al., Leukemia, 32(8): 1768-1777 (2018) (PMID: 30030507) |
| CD20 | Rituximab | Zhang et al., Signal Transduct. Target Ther., 1: 16002 (2016) (PMID: |
| | | 29263894) |
| | Leu 16 | Lee et al., J. Immunother., 41(1): 19-31 (2018) (PMID: 29176334) |
| | CD20 scFvs | International Patent Application Publication No. WO/2016/164731 |
| BCMA | BCMA-02 | Raje et al., N. Engl. J. Med., 380(18): 1726-1737 (2019) (PMID: |
| | | 31042825) |
| | LCAR38 | Zhao et al., J. Hematol. Oncol., 11(1): 141 (2018) (PMID: 30572922) |
| | BCMA scFv | Smith et al., Cancer Immunol. Res., 7(7): 1047-1053 (2019) (PMID: |
| | | 31113804) |
| | Biepitopic | Xu et al., Proc. Natl. Acad. Sci. USA, 116(19): 9543-9551 (2019) |
| | | (PMID: 30988175) |
| | NVS BCMA | Cohen et al., J. Clin. Invest., 129(6): 2210-2221 (2019) (PMID: |
| | | 30896447) |
| CS-1 | CS1R | Wang et al., Clin. Cancer Res., 24(1): 106-119 (2018) (PMID: |
| | | 29061640) |
| | CS1 ScFv | Chu et al., Leukemia, 28(4): 917-27 (2014) (PMID: 24067492) |
| | Elotuzumab based | Dimopoulos et al., N. Engl. J. Med., 379(19): 1811-1822 (2018) (PMID: |
| | | 30403938) |
| CD138 | ScFv | Sun et al., Oncotarget., 10(24): 2369-2383 (2019) (PMID: 31040928) |
| CD44v6 | Humanized scFv | Leuci et al., Oncoimmunology, 7(5): e1423167 (2018) (PMID: |
| | | 29721373) |
| | cMAb U36 | Sandstrom et al., Int. J. Oncol., 40(5): 1525-32 (2012) (PMID: |
| | | 22307465) |
| NKG2D | scFv | Yang et al., J. Immunother. Cancer, 7(1): 171 (2019) (PMID: 31288857) |
| | NKG2Dg scFv | Parihar et al., Cancer Immunol. Res., 7(3): 363-375 (2019) (PMID: |
| | | 30651290) |
| CD38 | Nanobody CD38 | An et al., Mol. Pharm., 15(10): 4577-4588 (2018) (PMID: 30185037) |
| | Humanized scFv | Yoshida et al., Clin. Transl. Immunology, 5(12): e116 (2016) (PMID: |
| | | 28090317) |
| GPRC5D | Humanized scFv | Smith et al., Sci. Transl. Med., 11(485)eaau7746 (2019) (PMID: |
| | | 30918115) |
| CD79b | scFv (L-H) and (H-L) | Ormhoj et al., Clin. Cancer Res., (2019) (PMID: 31439577) |
| CD103 | M290 | Zhang et al., Am. J. Transplant., 9(9): 2012-23 (2009) (PMID: |
| | | 19645708) |
| FAP | FAP5 | Wang et al., Cancer Immunol. Res., 2(2): 154-66 (2014) (PMID: |
| | | 24778279) |
| CD70 | Human CD70 | Park et al., Oral Oncol., 78: 145-150 (2018) (PMID: 29496042) |
| MUC16 | 4H11 | Koneru et al., Oncoimmunol., 4(3): e994446 (2015) (PMID: 25949921) |
| IL13Ra2 | IL13R | Kong et al., Clin. Cancer Res., 18(21): 5949-60 (2012) (PMID: |
| | | 22966020) |

Any appropriate method can be used to express an antigen receptor on a T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide. For example, a nucleic acid encoding an antigen receptor can be introduced into one or more T cells. In some cases, viral transduction can be used to introduce a nucleic acid encoding an antigen receptor into a non-dividing a cell. A nucleic acid encoding an antigen receptor can be introduced in a T cell using any appropriate method. In some cases, a nucleic acid encoding an antigen receptor can be introduced into a T cell by transduction (e.g., viral transduction using a retroviral vector such as a lentiviral vector) or transfection. In some cases, a nucleic acid encoding an antigen receptor can be introduced ex vivo into one or more T cells. For example, ex vivo engineering of T cells expressing an antigen receptor can include transducing isolated T cells with a lentiviral vector encoding an antigen receptor. In cases where T cells are engineered ex vivo to express an antigen receptor, the T cells can be obtained from any appropriate source (e.g., a mammal such as the mammal to be treated or a donor mammal, or a cell line).

In some cases, when a T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide also expresses (e.g., is engineered to express) an antigen receptor, that T cell can be engineered to have a reduced level of an IL-4 polypeptide, a reduced level of a TCF7 polypeptide, a reduced level of a PTPN2 polypeptide, and/or a reduced level of a PTPN3 polypeptide and engineered to express an antigen receptor using any appropriate method. In some cases, a T cell can be engineered to have a reduced level of an IL-4 polypeptide, a reduced level of a TCF7 polypeptide, a reduced level of a PTPN2 polypeptide, and/or a reduced level of a PTPN3 polypeptide first and engineered to express an antigen receptor second, or vice versa. In some cases, a T cell can be simultaneously engineered to have a reduced level of an IL-4 polypeptide, a reduced level of a TCF7 polypeptide, a reduced level of a PTPN2 polypeptide, and/or a reduced level of a PTPN3 polypeptide and to express an antigen receptor. For example, one or more nucleic acids used to reduce a level of an IL-4 polypeptide, reduce a level of a TCF7 polypeptide, reduce a level of a PTPN2 polypeptide, and/or reduce a level of a PTPN3 polypeptide (e.g., a lentiviral vector encoding a nucleic acid molecule designed to induce RNA interference and/or a lentiviral vector encoding gene-editing components) and one or more nucleic acids encoding an antigen receptor (e.g., a CAR) can be simultaneously introduced into one or more T cells. One or more nucleic acids used to reduce a level of an IL-4 polypeptide, reduce a level of a TCF7 polypeptide, reduce a level of a PTPN2 polypeptide, and/or reduce a level of a PTPN3 polypeptide, and one or more nucleic acids encoding an antigen receptor can be introduced into one or more T cells on separate nucleic acid constructs or on a single nucleic acid construct. In some cases, one or more nucleic acids used to reduce a level of an IL-4 polypeptide, reduce a level of a TCF7 polypeptide, reduce a level of a PTPN2 polypeptide, and/or reduce a level of a PTPN3 polypeptide, and one or more nucleic acids encoding an antigen receptor can be introduced into one or more T cells on a single nucleic acid construct. In some cases, one or more nucleic acids used to reduce a level of an IL-4 polypeptide, reduce a level of a TCF7 polypeptide, reduce a level of a PTPN2 polypeptide, and/or reduce a level of a PTPN3 polypeptide, and one or more nucleic acids encoding an antigen receptor can be introduced ex vivo into one or more T cells. In cases where T cells are engineered ex vivo to have a reduced level of an IL-4 polypeptide, a reduced level of a TCF7 polypeptide, a reduced level of a PTPN2 polypeptide, and/or a reduced level of a PTPN3 polypeptide, and to express an antigen receptor, the T cells can be obtained from any appropriate source (e.g., a mammal such as the mammal to be treated or a donor mammal, or a cell line).

In some cases, a T cell having (e.g., engineered to have) a reduced level of an IL-4 polypeptide, a reduced level of a TCF7 polypeptide, a reduced level of a PTPN2 polypeptide, and/or a reduced level of a PTPN3 polypeptide can be stimulated. A T cell can be stimulated at the same time as being engineered to have a reduced level of an IL-4 polypeptide, a reduced level of a TCF7 polypeptide, a reduced level of a PTPN2 polypeptide, and/or a reduced level of a PTPN3 polypeptide or independently of being engineered to have a reduced level of an IL-4 polypeptide, a reduced level of a TCF7 polypeptide, a reduced level of a PTPN2 polypeptide, and/or a reduced level of a PTPN3 polypeptide. For example, one or more T cells having a reduced level of an IL-4 polypeptide, a reduced level of a TCF7 polypeptide, a reduced level of a PTPN2 polypeptide, and/or a reduced level of a PTPN3 polypeptide used in an adoptive cell therapy can be stimulated first, and can be engineered to have a reduced level of an IL-4 polypeptide, a reduced level of a TCF7 polypeptide, a reduced level of a PTPN2 polypeptide, and/or a reduced level of a PTPN3 polypeptide second, or vice versa. In some cases, one or more T cells having a reduced level of an IL-4 polypeptide, a reduced level of a TCF7 polypeptide, a reduced level of a PTPN2 polypeptide, and/or a reduced level of a PTPN3 polypeptide used in an adoptive cell therapy can be stimulated first, and can be engineered to have a reduced level of an IL-4 polypeptide, a reduced level of a TCF7 polypeptide, a reduced level of a PTPN2 polypeptide, and/or a reduced level of a PTPN3 polypeptide second. A T cell can be stimulated using any appropriate method. For example, a T cell can be stimulated by contacting the T cell with one or more CD polypeptides. Examples of CD polypeptides that can be used to stimulate a T cell include, without limitation, CD3, CD28, inducible T cell co-stimulator (ICOS), CD137, CD2, OX40, CD27, MYD88, and CD40L.

This document also provides methods and materials involved in treating cancer. For example, one or more T cells having (e.g., engineered to have) a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) can be administered (e.g., in an adoptive cell therapy such as a CAR T cell therapy) to a mammal (e.g., a human) having cancer to treat the mammal. In some cases, methods of treating a mammal having cancer as described herein can reduce the number of cancer cells (e.g., cancer cells expressing a tumor antigen) within a mammal. In some cases, methods of treating a mammal having cancer as described herein can reduce the size of one or more tumors (e.g., tumors expressing a tumor antigen) within a mammal.

Any appropriate amount (e.g., number) of T cells having (e.g., engineered to have) a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) can be administered (e.g., in an adoptive cell therapy such as a CAR T cell therapy) to a mammal (e.g., a human) having cancer. In some cases, from about 0.1×10⁶ T cells (e.g., CAR T cells) to about 10×10⁶ T cells (e.g., CAR T cells) having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) per killigram (kg) body weight of the mammal (e.g., from about 0.1×10⁶ to about 9×10⁶, from about 0.1×10⁶ to about 8×10⁶, from about 0.1×10⁶ to about 7×10⁶, from about 0.1×10⁶ to about 6×10⁶, from about 0.1×10⁶ to about 5×10⁶, from about 0.1×10⁶ to about 4×10⁶, from about 0.1×10⁶ to about 3×10⁶, from about 0.1×10⁶ to about 2×10⁶, from about 0.1×10⁶ to about 1×10⁶, from about 0.1×10⁶ to about 0.5×10⁶, from about 0.5×10⁶ to about 10×10⁶, from about 1×10⁶ to about 10×10⁶, from about 2×10⁶ to about 10×10⁶, from about 3×10⁶ to about 10×10⁶, from about 4×10⁶ to about 10×10⁶, from about 5×10⁶ to about 10×10⁶, from about 6×10⁶ to about 10×10⁶, from about 7×10⁶ to about 10×10⁶, from about 8×10⁶ to about 10×10⁶, from about 9×10⁶ to about 10×10⁶, from about 0.5×10⁶ to about 9×10⁶, from about 1×10⁶ to about 8×10⁶, from about 2×10⁶ to about 7×10⁶, from about 3×10⁶ to about 6×10⁶, from about 4×10⁶ to about 5×10⁶, from about 1×10⁶ to about 3×10⁶, from about 2×10⁶ to about 4×10⁶, from about 3×10⁶ to about 5×10⁶, from about 4×10⁶ to about 6×10⁶, from about 5×10⁶ to about 7×10⁶, from about 6×10⁶ to about 8×10⁶, or from about 7×10⁶ to about 9×10⁶ of T cells per kg) can be administered to a mammal having cancer to treat the mammal.

Any appropriate mammal (e.g., a human) having a cancer can be treated as described herein. Examples of mammals that can be treated as described herein include, without limitation, humans, non-human primates (e.g., monkeys), dogs, cats, horses, cows, pigs, sheep, mice, and rats. For example, a human having a cancer can be treated with one or more T cells having (e.g., engineered to have) a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) in, for example, an adoptive T cell therapy such as a CAR T cell therapy using the methods and materials described herein.

When treating a mammal (e.g., a human) having a cancer as described herein, the cancer can be any appropriate cancer. In some cases, a cancer treated as described herein can include one or more solid tumors. In some cases, a cancer treated as described herein can be a hematological (blood) cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. In some cases, a cancer treated as described herein can express a tumor-associated antigen (e.g., an antigenic substance produced by a cancer cell). Examples of cancers that can be treated as described herein include, without limitation, diffuse large B cell lymphoma (DLBCL), Hodgkin's lymphomas, non-Hodgkin lymphomas, acute lymphoblastic leukemia (ALL), chronic lymphocytic leukemia (CLL), acute myeloid leukemia (AML), germ cell tumors, hepatocellular carcinoma, bowel cancer, lung cancer, breast cancer, ovarian cancer, melanoma, epithelial tumors, brain cancers, multiple myelomas, sarcomas, bone cancers, pancreatic cancers, liver cancers, myeloid neoplasms, prostate cancers, and thyroid cancers.

In some cases, the methods described herein can include identifying a mammal (e.g., a human) as having a cancer. Any appropriate method can be used to identify a mammal having cancer. For example, imaging techniques and biopsy techniques can be used to identify mammals (e.g., humans) having cancer.

In some cases, the methods described herein can include identifying a mammal (e.g., a human) as being in need of T cells (e.g., CAR T cells) having reduced susceptibility to T cell (e.g., CAR T cell) cell exhaustion. Any appropriate method can be used to identify a mammal as being in need of T cells (e.g., CAR T cells) having reduced susceptibility to T cell (e.g., CAR T cell) cell exhaustion. For example, medical histories (e.g., evaluations of disease state and/or knowledge of response to prior therapies) and/or diagnosis (e.g., diagnosis with a cancer that is difficult to treat such as hematological cancer that is difficult to treat) can be used to identify mammals (e.g., humans) as being in need of such T cells (e.g., such CAR T cells).

A mammal (e.g., a human) having a cancer can be administered one or more T cells having (e.g., engineered to have) a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) described herein. For example, one or more T cells having (e.g., engineered to have) a reduced level of an IL-4 polypeptide, a reduced level of a TCF7 polypeptide, a reduced level of a PTPN2 polypeptide, and/or a reduced level of a PTPN3 polypeptide can be used in an adoptive T cell therapy (e.g., a CAR T cell therapy) to treat a mammal having a cancer. For example, one or more T cells having a reduced level of an IL-4 polypeptide, a reduced level of a TCF7 polypeptide, a reduced level of a PTPN2 polypeptide, and/or a reduced level of a PTPN3 polypeptide can be used in an adoptive T cell therapy (e.g., a CAR T cell therapy) targeting any appropriate antigen within a mammal (e.g., a mammal having cancer). In some cases, an antigen can be a tumor-associated antigen (e.g., an antigenic substance produced by a cancer cell). Examples of tumor-associated antigens that can be targeted by an adoptive T cell therapy provided herein include, without limitation, CD19 (associated with DLBCL, ALL, and CLL), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), MAGE (associated with malignant melanoma), CD33 (associated with AML), CD123 (associated with AML), CLL-1 (associated with AML), E-Cadherin (associated with epithelial tumors), folate receptor alpha (associated with ovarian cancers), folate receptor feta (associated with ovarian cancers and AML), IL13R (associated with brain cancers), EGFRviii (associated with brain cancers), CD22 (associated with B cell cancers), CD20 (associated with B cell cancers), kappa light chain (associated with B cell cancers), lambda light chain (associated with B cell cancers), CD44v (associated with AML), CD45 (associated with hematological cancers), CD30 (associated with Hodgkin lymphomas and T cell lymphomas), CD5 (associated with T cell lymphomas), CD7 (associated with T cell lymphomas), CD2 (associated with T cell lymphomas), CD38 (associated with multiple myelomas and AML), BCMA (associated with multiple myelomas), CD138 (associated with multiple myelomas and AML), FAP (associated with solid tumors), CS-1 (associated with multiple myeloma), TSHR (associated with thyroid cancers), and c-Met (associated with breast cancer). For example, one or more T cells having a reduced level of an IL-4 polypeptide, a reduced level of a TCF7 polypeptide, a reduced level of a PTPN2 polypeptide, and/or a reduced level of a PTPN3 polypeptide can be used in CAR T cell therapy targeting CD19 (e.g., a CART19 cell therapy) to treat cancer as described herein.

In some cases, one or more T cells having (e.g., engineered to have) a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) used in an adoptive T cell therapy (e.g., a CAR T cell therapy) can be administered to a mammal having a cancer as a combination therapy with one or more additional agents used to treat a cancer. For example, one or more T cells having a reduced level of an IL-4 polypeptide, a reduced level of a TCF7 polypeptide, a reduced level of a PTPN2 polypeptide, and/or a reduced level of a PTPN3 polypeptide used in an adoptive cell therapy can be administered to a mammal in combination with one or more anti-cancer treatments (e.g., surgery, radiation therapy, chemotherapy (e.g., alkylating agents such as busulfan). In cases where one or more T cells having a reduced level of an IL-4 polypeptide, a reduced level of a TCF7 polypeptide, a reduced level of a PTPN2 polypeptide, and/or a reduced level of a PTPN3 polypeptide used in an adoptive cell therapy are used with additional agents treat a cancer, the one or more additional agents can be administered at the same time or independently. In some cases, one or more T cells having a reduced level of an IL-4 polypeptide, a reduced level of a TCF7 polypeptide, a reduced level of a PTPN2 polypeptide, and/or a reduced level of a PTPN3 polypeptide used in an adoptive cell therapy can be administered first, and the one or more additional agents administered second, or vice versa.

In some cases, the methods and materials described herein can be applied to immune cells other than T cells, such as natural killer (NK) cells. For example, the methods and materials described herein can be used to design NK cells having (e.g., engineered to have) a reduced level of an IL-4 polypeptide (e.g., IL-4 KO CAR-NK cells), a reduced level of a TCF7 polypeptide (e.g., TCF7 KO CAR-NK cells), a reduced level of a PTPN2 polypeptide (e.g., PTPN2 KO CAR-NK cells), and/or a reduced level of a PTPN3 polypeptide (e.g., PTPN3 KO CAR-NK cells). In some cases, NK cells having a reduced level of an IL-4 polypeptide, a reduced level of a TCF7 polypeptide, a reduced level of a PTPN2 polypeptide, and/or a reduced level of a PTPN3 polypeptide can be used in an adoptive cell therapy (e.g., a CAR-NK cell therapy). For example, NK cells having a reduced level of an IL-4 polypeptide, a reduced level of a TCF7 polypeptide, a reduced level of a PTPN2 polypeptide, and/or a reduced level of a PTPN3 polypeptide can be administered to a mammal having a cancer to treat the cancer. For example, NK cells having a reduced level of an IL-4 polypeptide, a reduced level of a TCF7 polypeptide, a reduced level of a PTPN2 polypeptide, and/or a reduced level of a PTPN3 polypeptide can be administered to a mammal having a cancer as a combination therapy with one or more additional agents used to treat a cancer.

The invention will be further described in the following examples, which do not limit the scope of the invention described in the claims.

### EXAMPLES

### Example 1: Induction of CAR T Cell Exhaustion

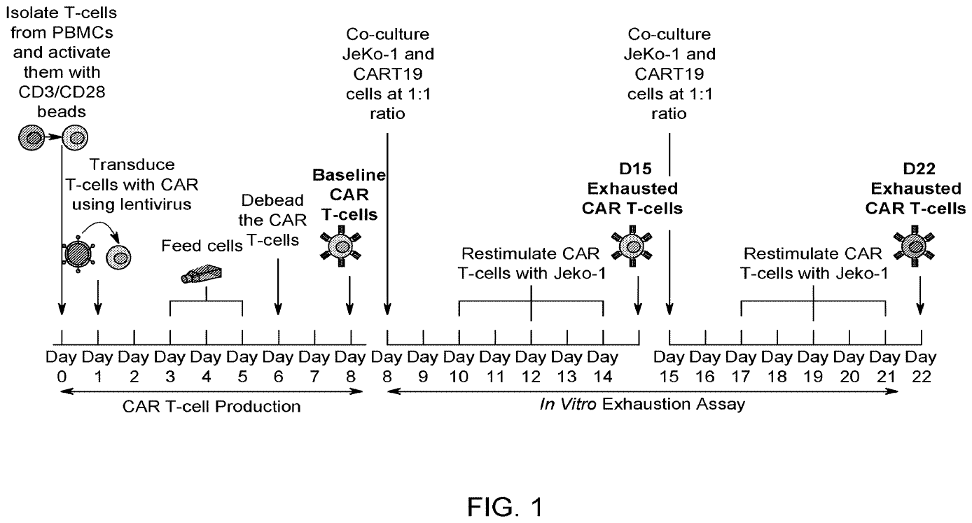

An in vitro assay to induce antigen-specific exhaustion in CAR T cells was developed as shown in FIG. 1.

This assay incorporated a CAR T cell production period of 8 days. On day 0 of the production period, T cells were isolated from peripheral blood mononuclear cells (PBMCs) and stimulated with CD3/CD28 beads using a 3:1 ratio of beads to cells. Then, on day 1, the activated T cells were transduced with CAR using lentivirus at a multiplicity of infection of 3. These cells were maintained at a concentration of 1 million cells/mL from day 0 to day 6. On day 6, the CD3/CD28 beads were removed with magnetic separation, CAR expression was measured by staining the cells with a antibody against ProteinL and using flow cytometry to quantify the percent of positively stained cells. The cells were then rested from day 6 to day 8. On day 8, cells were considered baseline CAR T cells.

For the exhaustion assay, day 8 CART19 cells were stimulated at a 1 to 1 ratio with the CD19-expressing tumor cell line, JeKo-1. To maintain antigen-specific stimulation of the CART19 cells, the same number of JeKo-1 cells as was added on day 8 was added every other day (on day 10, day 12, and day 14). On day 15, the CART19 cells were considered exhausted, but the assay was continued until day 22 to establish more terminally exhausted CAR T cells.

Proliferation assays and degranulation assays were conducted using day 8 (baseline/unstimulated CAR T cells), day 15 (one-week exhausted CAR T cells), or day 22 (two-week exhausted CAR T cells).

### Proliferation Assay

Day 0 (start of the proliferation assay using day 8, day 15, or day 22 cells from the exhaustion assay):
   In a 96-well plate, combine 100 μL of 1×10⁶ cells/mL effector cells (CART19 cells) with 100 L of 1×10⁶ cells/mL target cells (JeKo-1 cells).
Day 3—Feed the cells with fresh media:
   Remove 100 μL of supernatant from each well
   Add 100 μL of T cell media (TCM) to ach well and resuspend
Day 5: Use flow cytometry to detect live CD3+ cells.

### Degranulation Assay

Using either day 8, day 15, or day 22 cells, plate the degranulation assay as follows:
   Add the following to each well:
      3 μL CD107a FITC (markers of immune cell activation and degranulation)
      1 μL monesin (secretion inhibitor)
      1 μL CD49d (antibody for costimulation)
      2 μL CD28 (antibody for costimulation)
   3 μL Flow Buffer
   Add 100 μL of targets (JeKo-1 cells) at 5×10⁶ cells/mL+100 μL of CART19 cells at 1×10⁶ cells/mL
Incubate the cell mixture for 4 hours
Use flow cytometry to detect the following: CD3, CD4, CD8, Live/Dead, IL-2, TNF-α, IFN-γ, and GM-CSF.

### Measurement of Inhibitory Receptors:

On days 8, 15, and 22 of the in vitro exhaustion assay, the ratio of CD4⁺ to CD8⁺ T cells and the percent of T cells that express an inhibitory receptor was determined by staining cells with antibodies for Live/Dead, CD3, CD8, PD-1, TIM-3, CTLA-4, and LAG-3. The parent population for these measurements was live CD3⁺ cells.

### Repeated Stimulation Assay

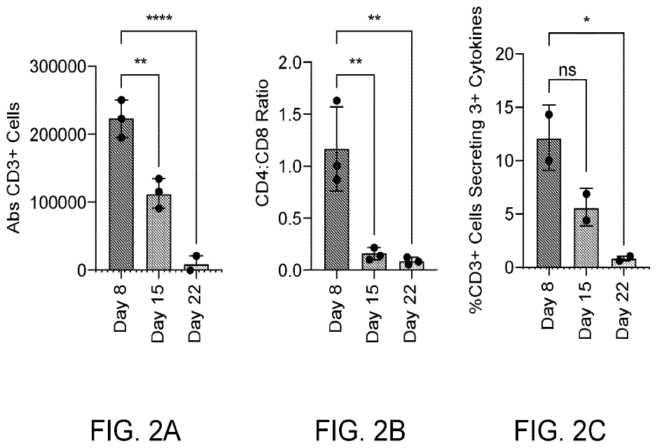

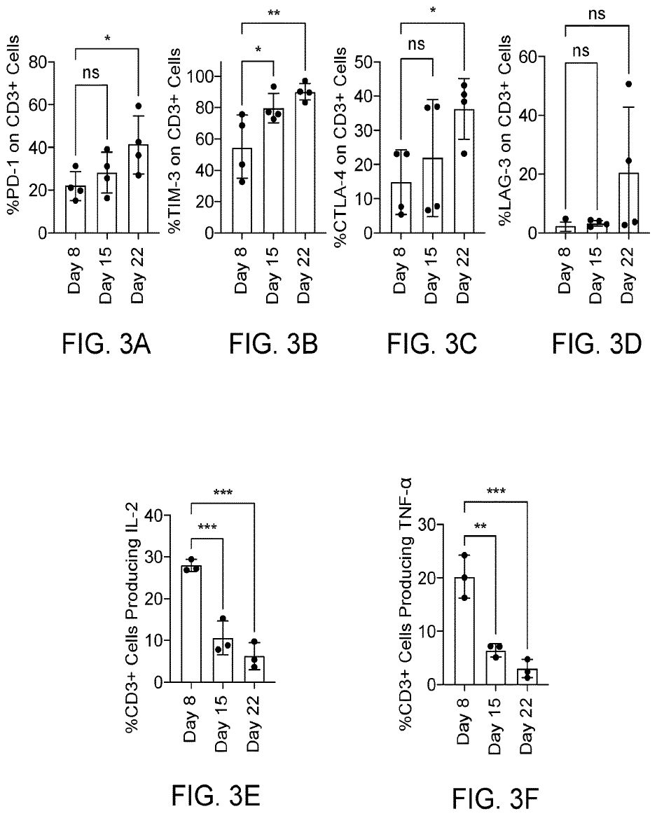

To model antigen-driven exhaustion, a 14-day in vitro repeated stimulation assay was developed with healthy donor CART19 cells with a CD28ζ costimulatory domain (CART19-28ζ) (FIG. 1). On Day 15, after 7 days of repeated CAR stimulation (with the CD19⁺ tumor cell line JeKo-1), CART19-28ζ cells demonstrated signs of general dysfunction such as a decrease in antigen-specific proliferative ability (FIG. 2A) and a decrease in the ratio of CD4⁺ to CD8⁺ T cells (FIG. 2B). These signs of dysfunction became more severe as the CAR T cells were repeatedly stimulated for another week to generate Day 22 CART19-28ζ cells. In addition, there was a significant loss of polyfunctionality, the ability of CAR T cells to secrete 3 or more cytokines at once, by the Day 22 timepoint (FIG. 2C). The CART19-28ζ cells also began to show exhaustion-specific signs of dysfunction such as an increase in the expression of inhibitory receptors such as PD-1 (FIG. 3A), TIM-3 (FIG. 3B), CTLA-4 (FIG. 3C), and LAG-3 (FIG. 3D) on Days 15 and 22 of the in vitro exhaustion assay. Additionally, the production of effector cytokines IL-2 (FIG. 3E) and TNF-α (FIG. 3F) was by CART19-28ζ cells was reduced on Day 15 and 22 of the exhaustion assay as compared to Day 8 cells.

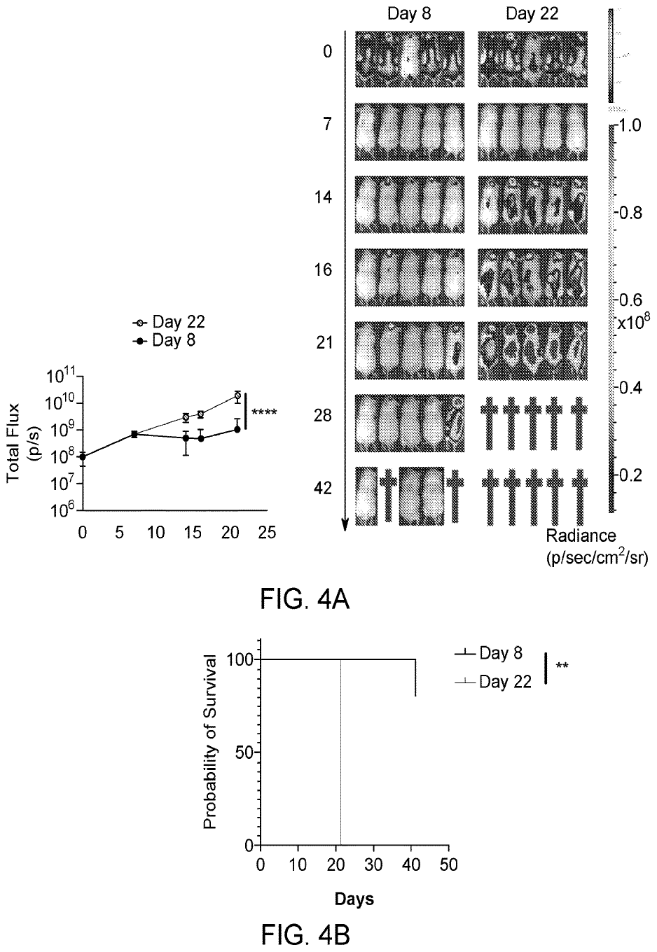

When CART19-28ζ cells from the exhaustion assay were tested using an in vivo JeKo-1 tumor model in NSG mice, Day 22 exhausted CART19-28ζ cells showed signs of dysfunction. For example, mice treated with Day 22 CART19-28ζ cells had higher tumor burdens (FIG. 4A) and they had reduced overall survival (FIG. 4B).

### Example 2: Epigenetic Landscape of T Cell Exhaustion

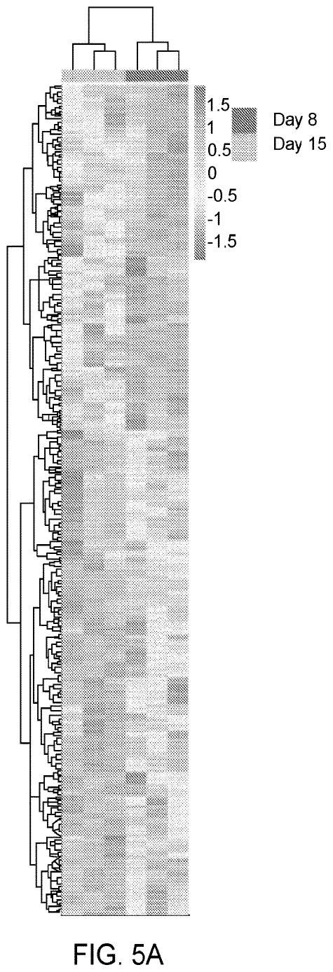

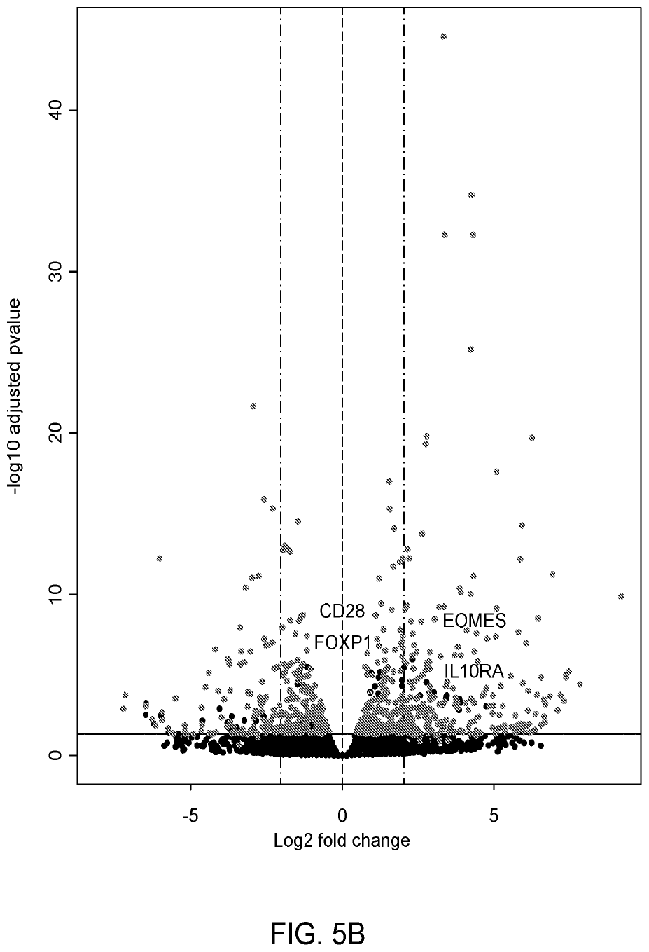

RNA and ATAC Sequencing of in vitro Exhausted CAR T Cells: To evaluate the transcriptomic and epigenetic profile of exhausted CAR T cells, RNA and ATAC sequencing was performed on Day 8 and Day 15 cells. An evaluation of the RNA sequencing data revealed the development of a distinct transcriptomic profile by Day 15 (FIG. 5A). Notably, known exhaustion genes, EOMES and IL10RA, were upregulated in the Day 15 cells (FIG. 5B).

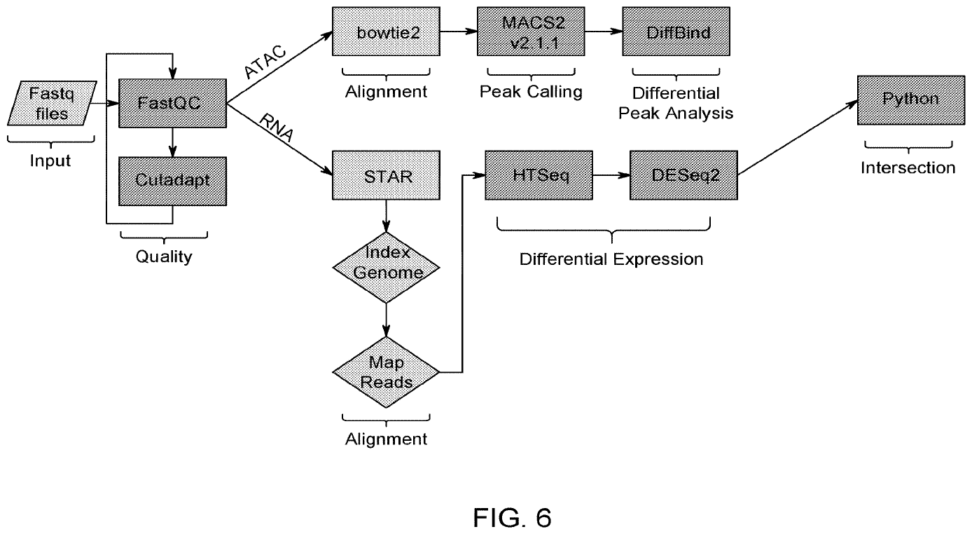

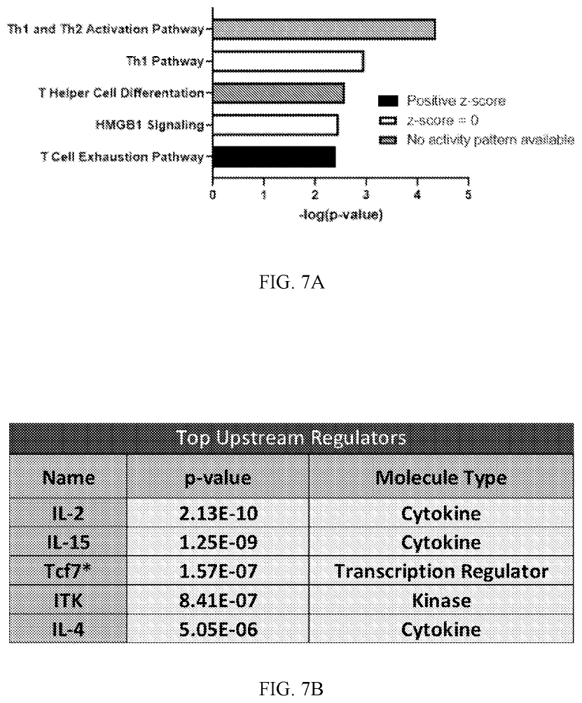

Next, to evaluate the genes that were both differentially expressed, as determined with RNA sequencing, and differentially accessible, as determined with ATAC sequencing, a sequencing analysis pipeline was developed to intersect the significant genes from each dataset (FIG. 6). RNA sequencing identified 449 significantly upregulated genes and 320 significantly downregulated genes in exhausted CART19-28ζ cells. ATAC sequencing identified 411 gene regions with increased accessibility and 445 gene regions with decreased accessibility. Overlap of RNA and ATAC results showed 105 genes that were both differentially expressed and differentially accessible. Analysis of the genes that were both differentially expressed and accessible with ingenuity pathway analysis revealed an enrichment in the T cell exhaustion pathway (p=4.23E-03, Z=1.0), suggested a potential role for the Th1 and Th2 activation pathway (p=4.43E-05) (FIG. 7A), and identified a regulatory role for molecules such as IL-2, Tcf-7, and IL-4 (FIG. 7B).

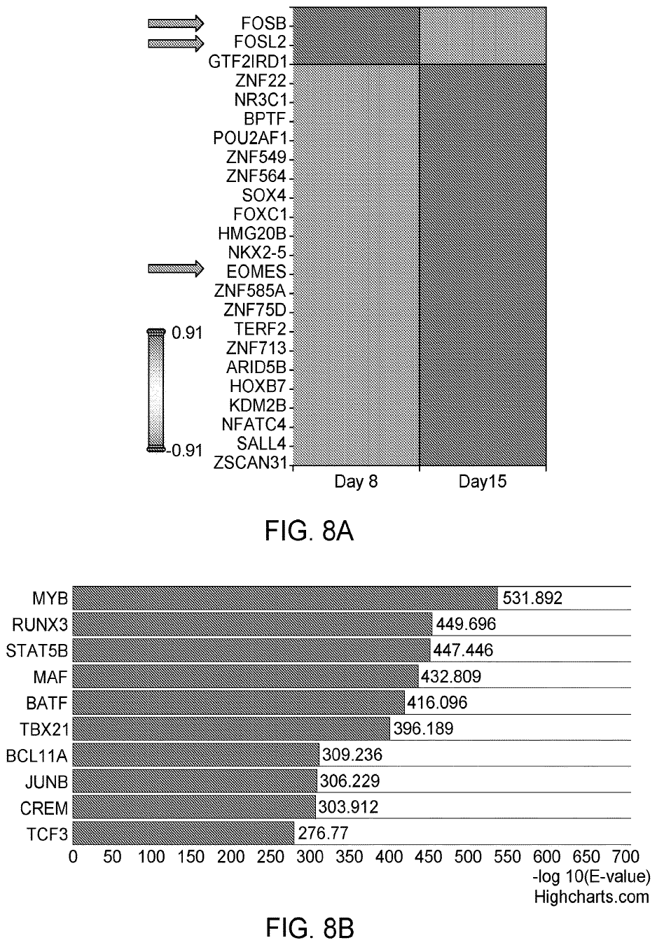

To look more specifically at transcription factors that mediate the development of exhaustion, an established multi-omics data analysis framework, Taiji, was used with the RNA and ATAC sequencing datasets. This analysis revealed an enrichment in the known exhausted-related transcription factor EOMES and a loss of AP-1 transcription factors FOSB and FOSL2 in the Day 15 samples (FIG. 8A). Additionally, mining of previously conducted ChIP sequencing experiments in T cells using the ReMAP database revealed an enrichment in binding sites for transcription factors such as TBX21, MYB (a regulator of Tcf-7), and RUNX3 (FIG. 8B).

RNA and ATAC Sequencing of Baseline Patient CAR T Cells from Responders and Non-Responders:

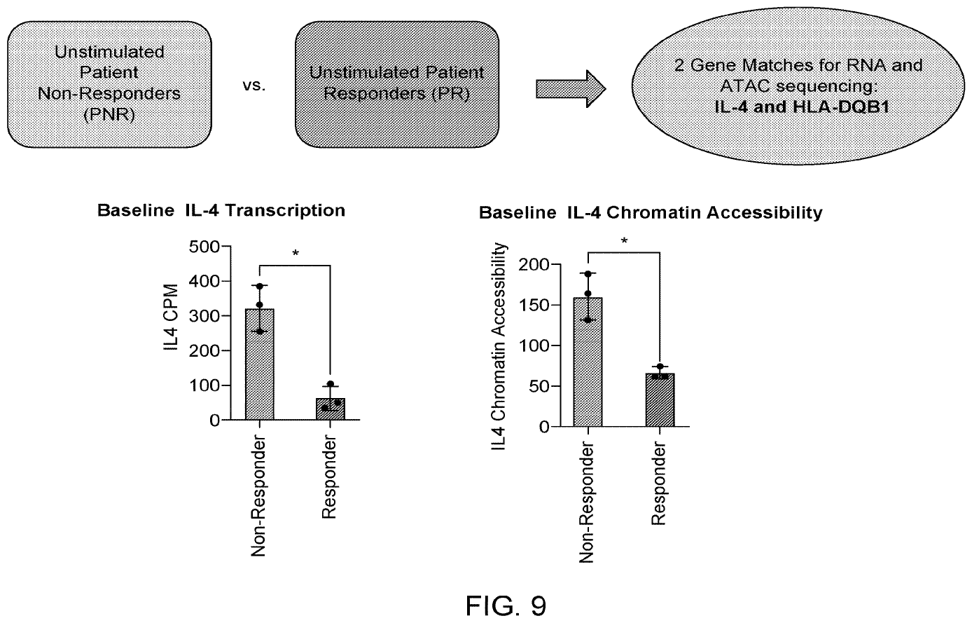

In order to evaluate CAR T cell dysfunction with a clinically relevant approach, RNA and ATAC sequencing was performed on pre-infusion axicabtagene ciloleucel (axi-cel; YESCARTA®) from 3 responders and 3 non-responders from the Zuma-1 clinical trial (Locke et al., Lancet Oncol., 20(1):31-42 (2019)). These products were CART19-28ζ cells that were generated from B cell lymphoma patient-derived T cells and sequenced after generation before infusion into the patient. RNA sequencing showed 54 differentially expressed genes and ATAC sequencing showed 24 differentially accessible gene regions. Only two genes, IL-4 and HLA-DQB1, were upregulated in non-responders based on both RNA and ATAC sequencing data (FIG. 9).

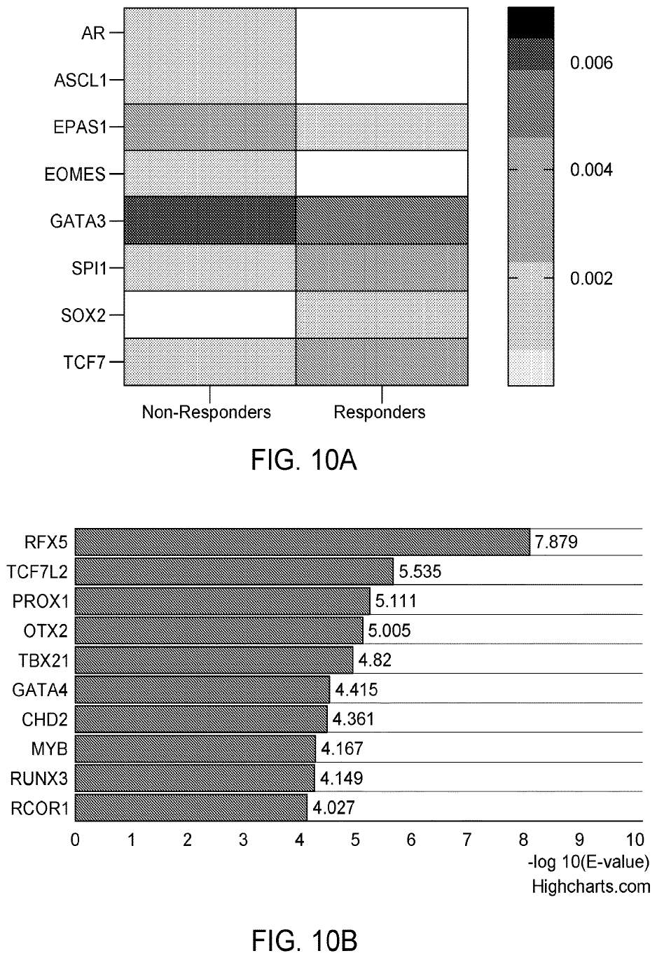

Next, to evaluate differences in the transcription factors enriched in baseline Axi-cel products from responders and non-responders, Taiji analysis was used. This analysis revelead an enrichment in EOMES and GATA3 in baseline products from non-responders (FIG. 10A). Additionally, mining of existing ChIPseq experiments with the ReMAP database again revealed an opening of binding sites for the transcription factors TBX21, MYB, and RUNX3 (FIG. 10B).

Comparison of RNA Sequencing Results from the In Vitro Exhaustion Assay and the Baseline Patient Products:

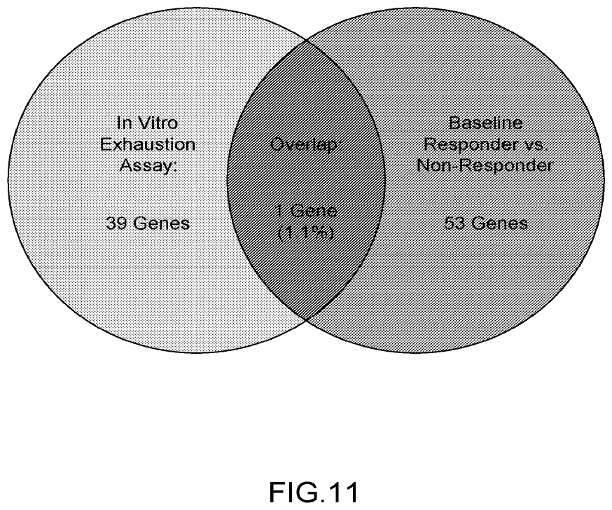

To evaluate similarities between the differential gene expression resulting from the in vitro exhaustion assay and the differential gene expression in baseline patient products from responders and non-responders, differentially expressed genes from the in vitro assay that were related to exhaustion and not activation were isolated. To do this, sequencing was also performed on Day 8 CAR T-cells that were stimulated with JeKo-1 cells at a 1:1 ratio for 24 hours. These CAR T-cells were referred to as Day Activated (DA) CAR T cells. It was discovered that 40 genes were differentially expressed when comparing both DA vs. Day 15 CAR T cells and when comparing Day 8 vs. Day 15 CAR T cells, but not when comparing Day 8 to DA CAR T cells. This list of 40 genes was then compared with the list of 54 genes that were differentially expressed between baseline products from patient responders and non-responders. Only one gene, PTPN3, was found to be in common between these two lists (FIG. 11).

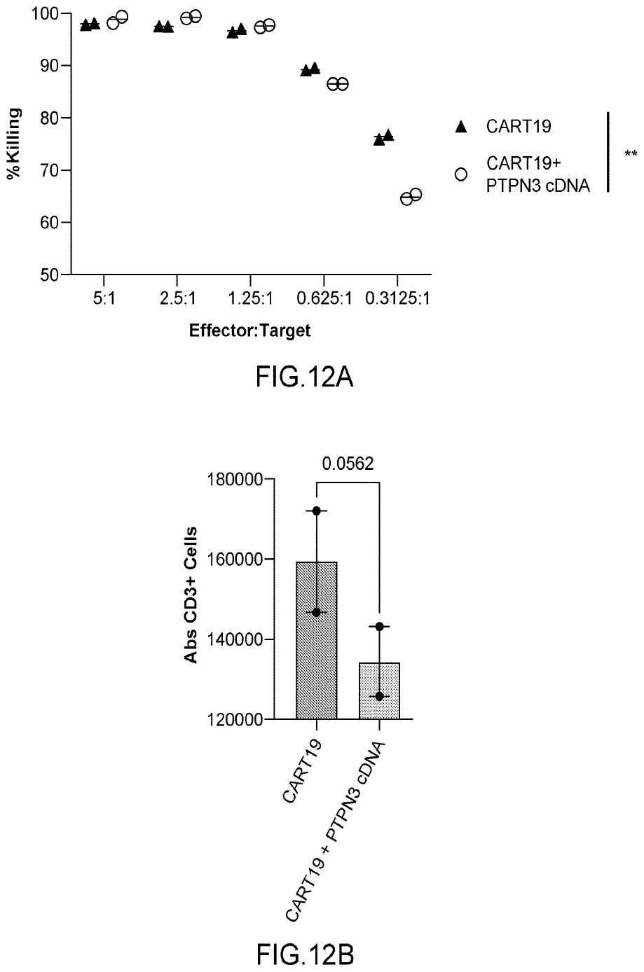

This indicates that PTPN3 may play a key role in the development of CAR T cell exhaustion related to the development of exhaustion. As such, in vitro validation experiments were performed by adding PTPN3 cDNA to CAR T cells and evaluating signs of dysfunction such as cytotoxicity and proliferation. CAR T cells that were exposed to PTPN3 cDNA exhibited reduced cytotoxicity (FIG. 12A) and reduced proliferative ability (FIG. 12B).

### Cytotoxicity Protocol

Day 0:
   Culture CART19 cells (effectors) with Jeko LucZsGr (targets) at the following E:T ratios (2.5:1, 1.25:1, 0.625:1, 0.312:1, 0.156:1, and 0:1)
Day 2: Measure 48 hr cytotoxicity:
   Dilute 200× luciferin 1:10 with sterile PBS
   Add 2 μL of diluted luciferin per well
   Resuspend the cells using a multichannel pipettor
   Check the luminescence with 10 second exposure

### Example 3: Conducting a Genome-Wide CRISPR Knockout Screen to Identify Key Genes in the Development of CAR T Cell Exhaustion

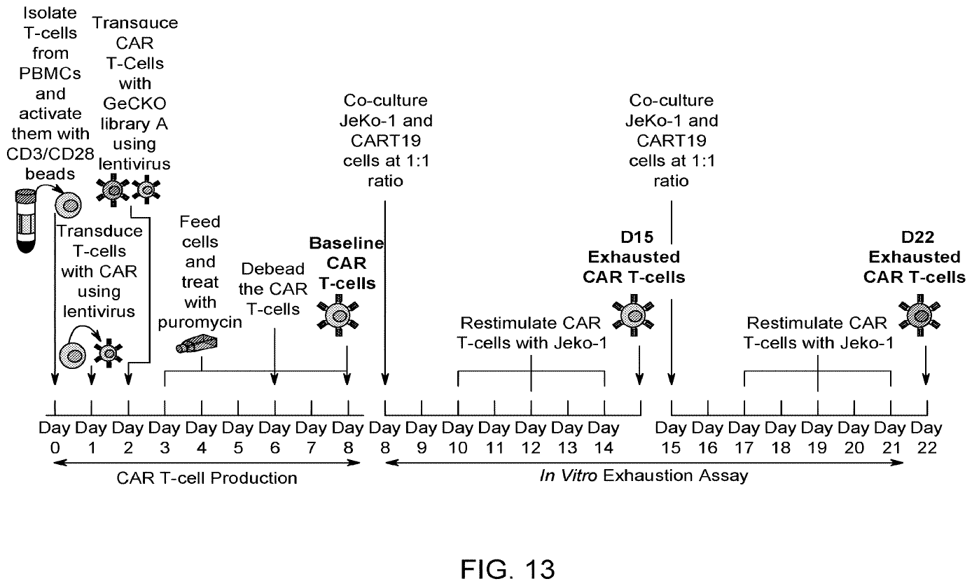

To further interrogate the epigenetic landscape of exhaustion in CART19-28ζ cells, a genome-wide CRISPR knockout screen in healthy donor CART19-28ζ cells was performed by utilizing an in vitro exhaustion assay (FIG. 13). For the screen, T cells experienced two lentiviral transduction events. The first event occurred on day 1 when the activated T cells were transduced with CAR lentivirus. Then, the second event occurred on day 2 when the CAR T cells were transduced with the gRNA library lentivirus. The gRNA library for this screen utilized a lentiCRISPRv2 vector that contained the gRNA sequence, Cas9 sequence, and a puromycin resistance cassette. The library consisted of 3 gRNAs per gene and 1,000 non-targeting gRNA sequences. Puromycin selection was performed from day 3 to day 8 by treating the CAR T cells with 1 g/ml puromycin. On day 8, samples were collected for next-generation sequencing (NGS) and then the remaining cells were stimulated with JeKo-1 cells at a 1:1 ratio as previously described in the in vitro exhaustion assay. On days 15 and 22, magnetic-activated cells sorting (MACS) was used to isolated CAR T-cells for NGS.

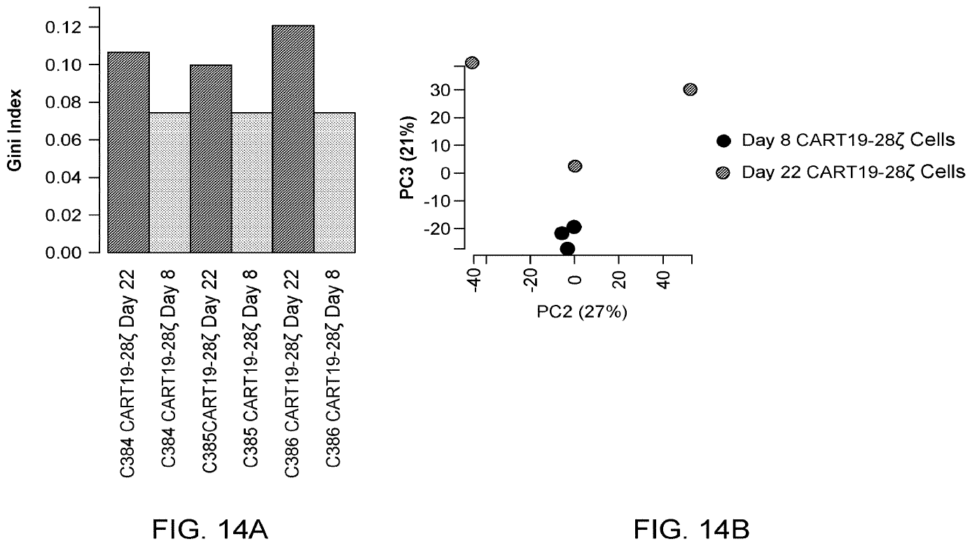

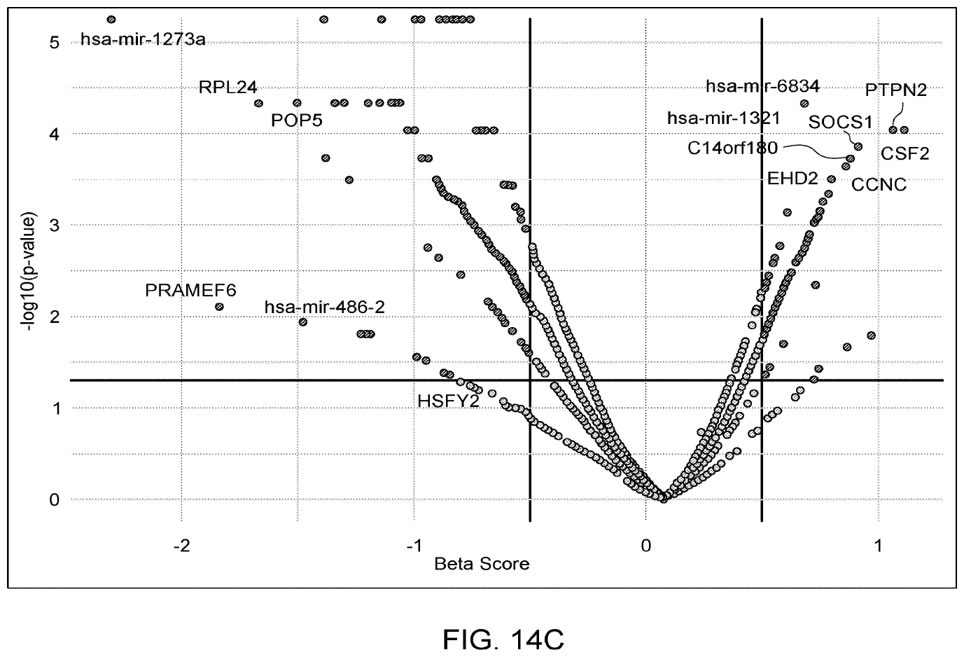

Analysis of the NGS results from the CRISPR screen with MAGeCK-VISPR, revealed successful positive selection by Day 22 of the screen (FIG. 14A). This is shown by an increase in the gini index, a measure of gRNA unevenness, from Day 8 to Day 22. Additionally, samples appeared to cluster due to condition (Day 8 vs. Day 22) as opposed to biological replicate (FIG. 14B). With the encouraging quality results from the CRISPR screen, genes that were positively or negatively selected for by generating a volcano plot were evaluated (FIG. 14C). Two of the top genes that were positively selected for in the screen include PTPN2 and CSF2. Thus, the depletion of PTPN2 and CSF2 appears to enhance CAR T Cell activity by enhancing their proliferative ability.

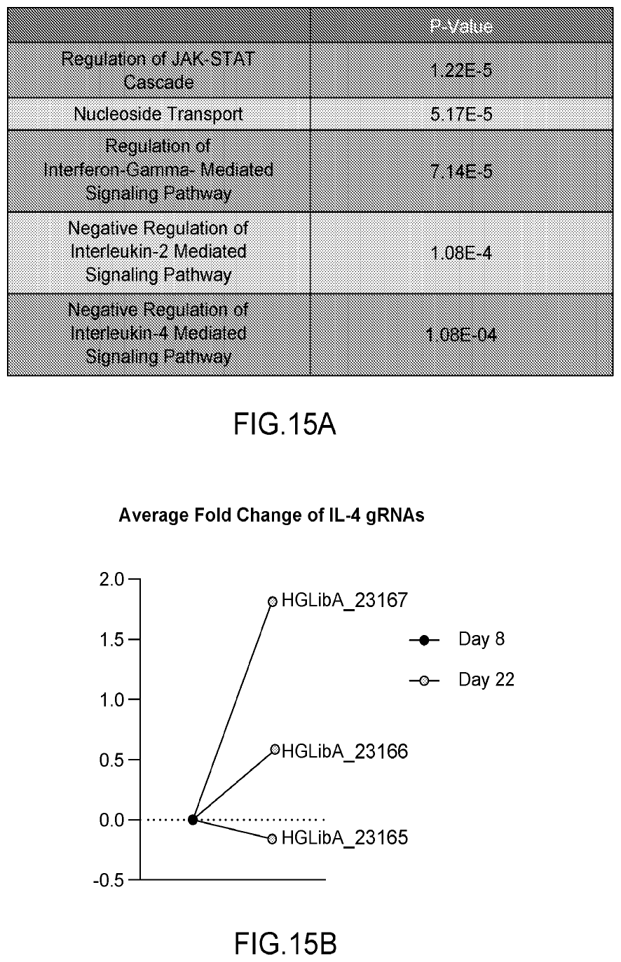

To further investigate the pathways that were positively selected for, gene ontology enrichment analysis was performed. The top pathways that were positively selected for include: the regulation of JAK-STAT cascade, negative regulation of IL-2 mediated signaling pathway, and negative regulation of IL-4 mediated signaling pathway (FIG. 15A). Investigation of the IL-4 in particular revealed positive selection of ⅔ gRNAs targeting IL-4 from Day 8 to Day 22 (FIG. 15B).

### Example 4: IL4 as a Regulator of CAR T-Cell Exhaustion

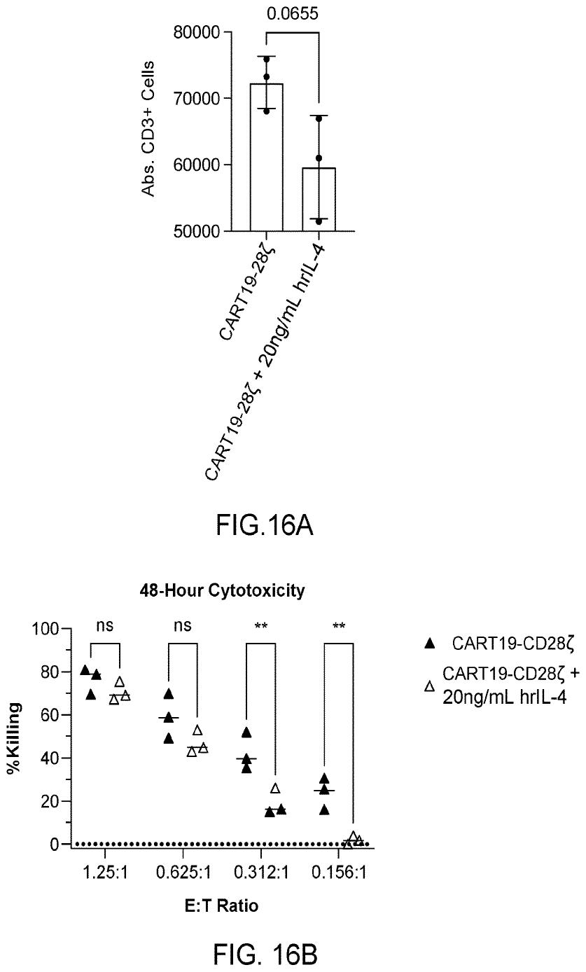

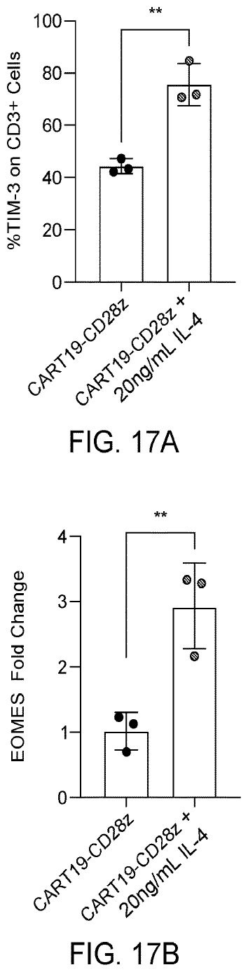

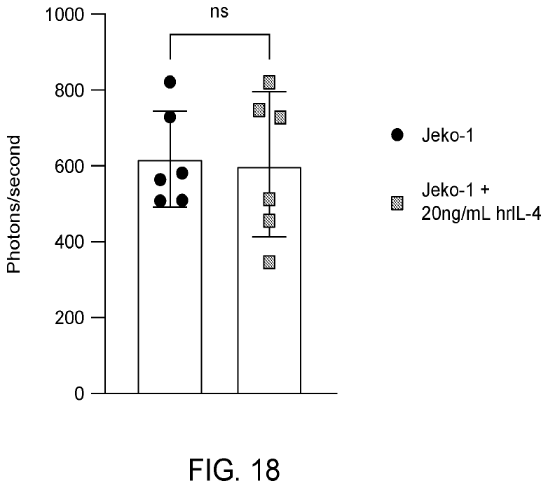

Since a regulatory role for IL-4 in CAR T-cell dysfunction was identified through three independent approaches: RNA and ATAC sequencing of baseline and exhausted CART19-28ζ cells from the in vitro exhaustion assay, RNA and ATAC sequencing of baseline Axi-cel products from responders and non-responders in the Zuma-1 clinical trial, and a genome-wide CRISPR knockout screen in healthy donor CART19-28ζ cells, additional in vitro and in vivo studies were performed to validate IL-4's role in dysfunction. To start, effector functions of healthy donor CART19-28ζ cells were assessed following treatment with human recombinant IL-4. Treatment of CART19-28ζ cells with 20 ng/mL human recombinant IL-4 (hrIL-4) resulted in general signs of CAR T-cell dysfunction such as a reduction in antigen specific proliferation (FIG. 16A) and a decrease in cytotoxicity (FIG. 16B). Additionally, treatment of CART19-28ζ cells with 20 ng/mL hrIL-4 resulted in exhaustion-specific signs of dysfunction by Day 15 such as an increase in the expression of the inhibitory receptor, TIM-3 (FIG. 17A), and an increase in the transcription of EOMES (FIG. 17B) as compared to CAR T-cells treated with diluent. IL-4 induced CART19-28ζ cell modulation was not due to a direct impact on tumor cells as identified by no change in tumor cytotoxicity or proliferation when JeKo-1 cells alone were treated with human recombinant IL-4 (FIG. 18). These data suggest that the IL-4 can regulate CAR T cell exhaustion that can lead to CART19 therapy failure.

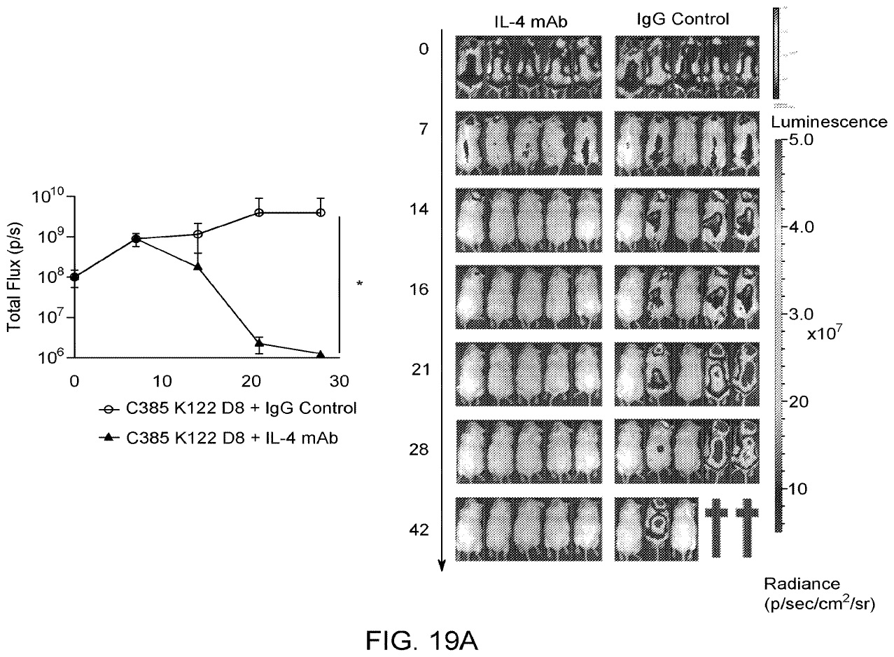

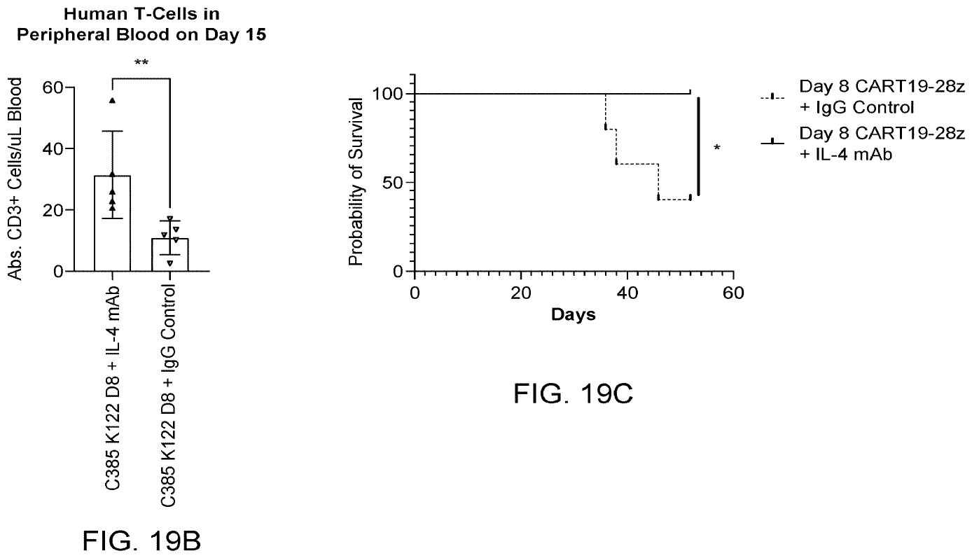

Next, to evaluate if IL-4 inhibition could prevent the development of exhaustion and therefore improve the efficacy of CAR T cell therapy, JeKo-1 xenograft NSG mice were treated with either a combination of Day 8 CART19-28ζ cells and 10 mg/kg IL-4 monoclonal antibody (mAb) or with a combination of Day 8 CART19-28ζ cells and 10 mg/kg IgG control antibody. Combination treatment with the IL-4 mAb significantly reduced tumor burden (FIG. 19A), increased CAR T cell proliferation (FIG. 19B), and increased overall survival (FIG. 19C). Thus, IL-4 inhibition can prevent CAR T cell dysfunction and improve overall treatment efficacy.

Taken together, these results demonstrate that IL-4 KO CAR T cells are less likely to undergo exhaustion (e.g., as compared to CAR T cells that are not engineered to KO a nucleic acid encoding an IL-4 polypeptide). Additional results of reducing IL-4 expression in T cells (e.g., CAR T cells) are shown in FIGS. 19-22.

### Example 5

The results in this Example re-present and expand on at least some of the results provided in other Examples.

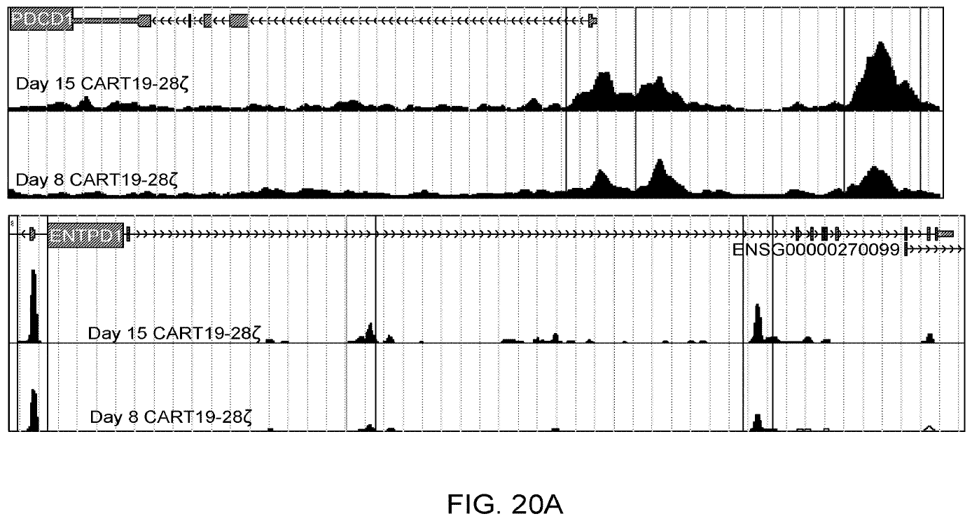

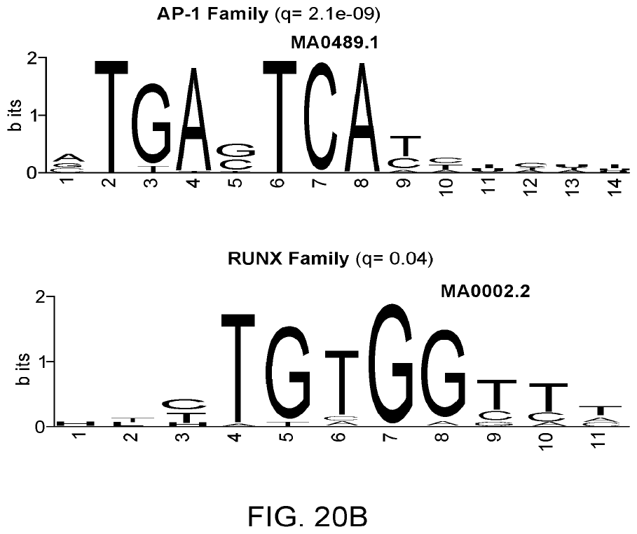

Since ATAC sequencing was also performed on Days 8 and 15 of the in vitro exhaustion assays, the epigenetic landscape of the baseline and chronically stimulated CAR T cells was evaluated. Consistent with the transcriptomic changes and existing literature, there was enhanced chromatin accessibility at exhaustion-related gene loci (e.g., PDCD1 and ENTPD1) in the chronically stimulated samples (FIG. 20A) as well as enhanced motif accessibility for AP-1 and RUNX family members (FIG. 20B).

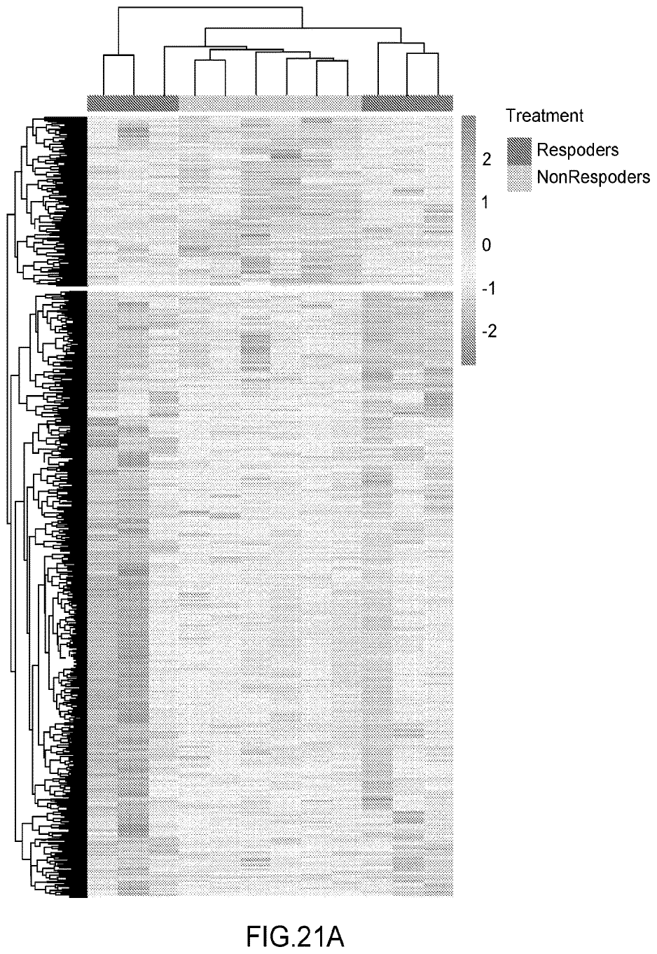

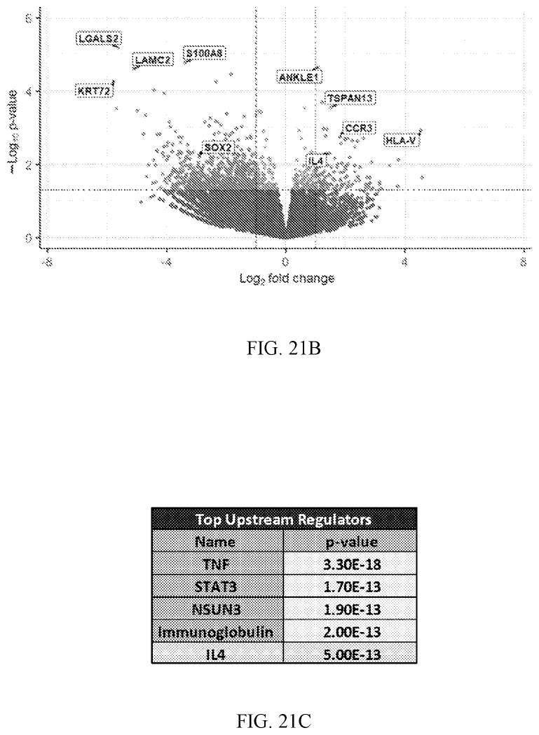

To evaluate CAR T-cell dysfunction in a more clinically relevant setting, RNA and ATAC sequencing was performed on baseline Axi-cel products from 6 responders and 6 non-responders from the Zuma-1 clinical trial. Interrogation of the transcriptome with RNA sequencing of pre-infusion axi-cel products from 6 responders and 6 non-responders showed clustering of non-responder samples (FIG. 21A). One of the top upregulated genes in CAR T cells from non-responders is IL-4 (FIG. 21B). Additionally, analysis of the differentially expressed genes with QIAGEN IPA identified IL-4 as one of the upstream regulators, along with other genes such as TNF and STAT3 (FIG. 21C) Differential gene analysis was performed with twelve biological replicates in total, six biological replicates per condition. A cut-off of p-value <0.05 was used for differentially expressed genes.

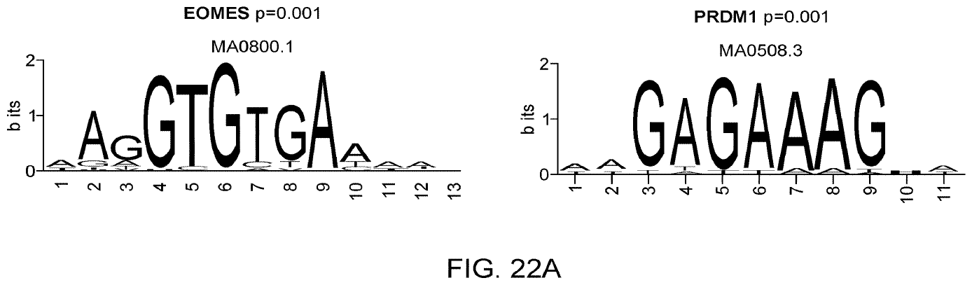

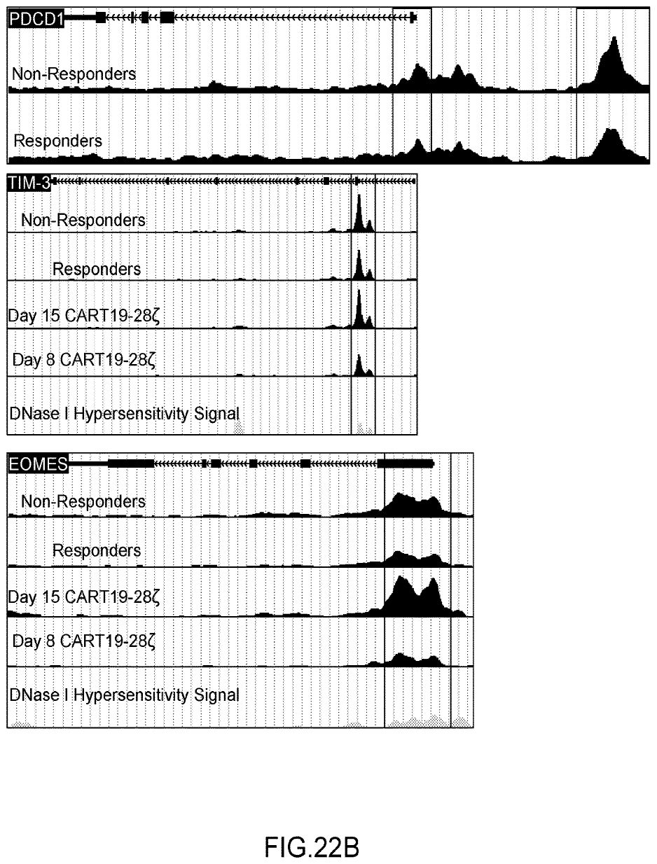

Analysis of chromatin changes between responder and non-responder samples revealed many similarities to the findings observed following chronic stimulation of healthy donor CART19-28ζ cells in our in vitro model for exhaustion. Motif analysis revealed an enrichment of motif binding sites for EOMES and PRDM1 (FIG. 22A). Additionally, non-responders showed similar enhancement of chromatin accessibility to exhausted cell populations at exhaustion-related gene loci such as PDCD1, HAVCR2 (TIM-3), and EOMES (FIG. 22B).

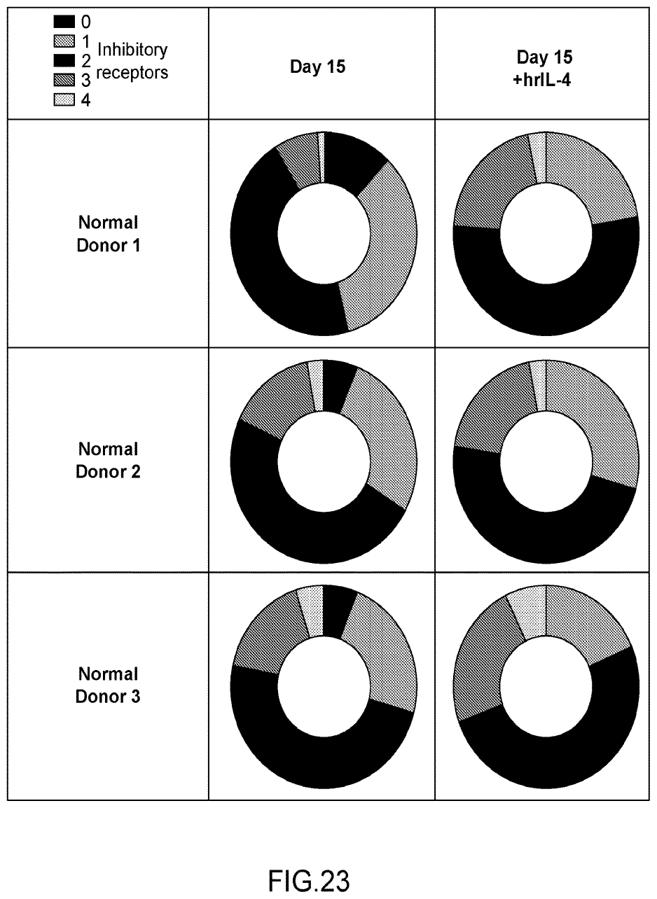

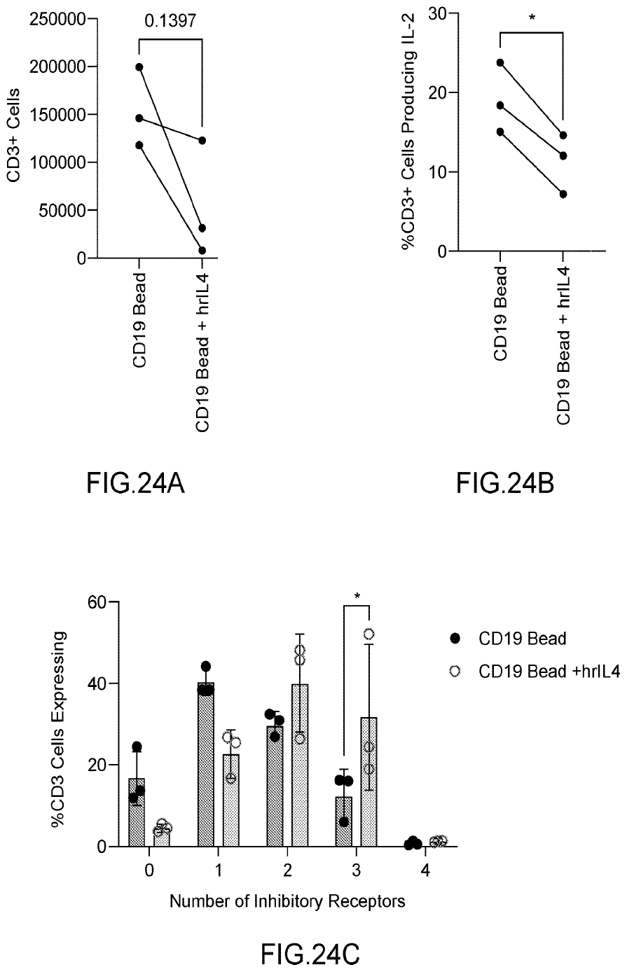

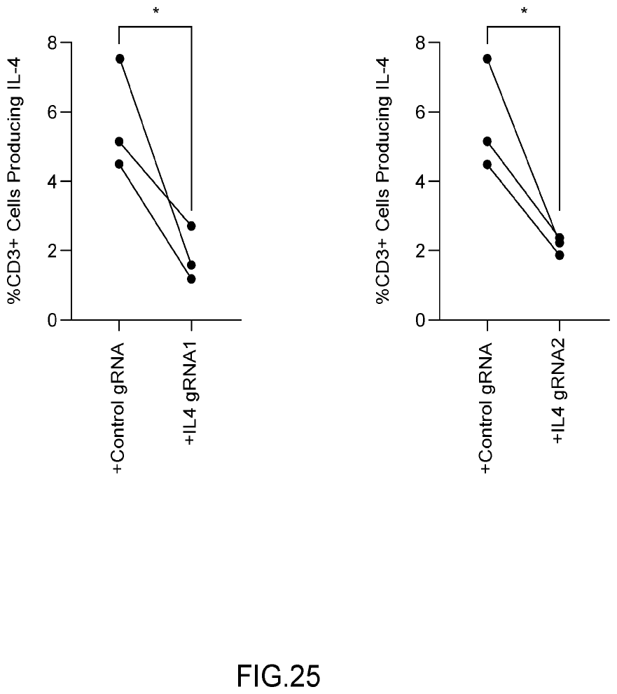

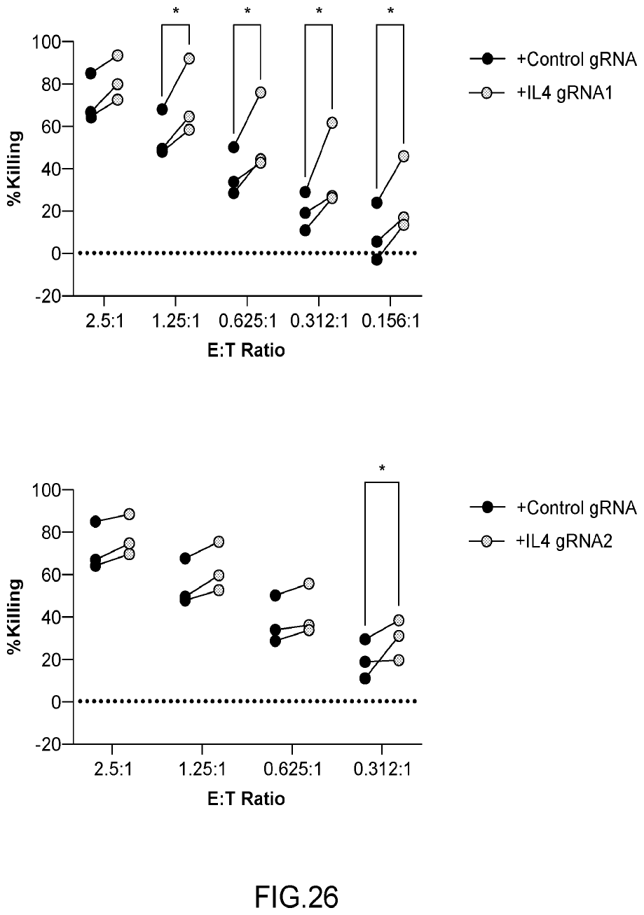

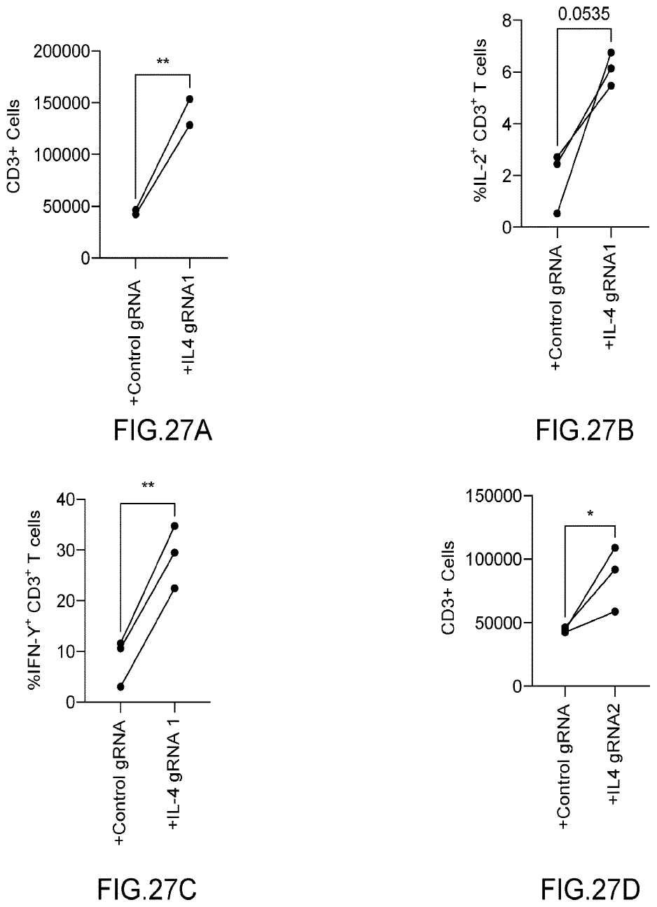

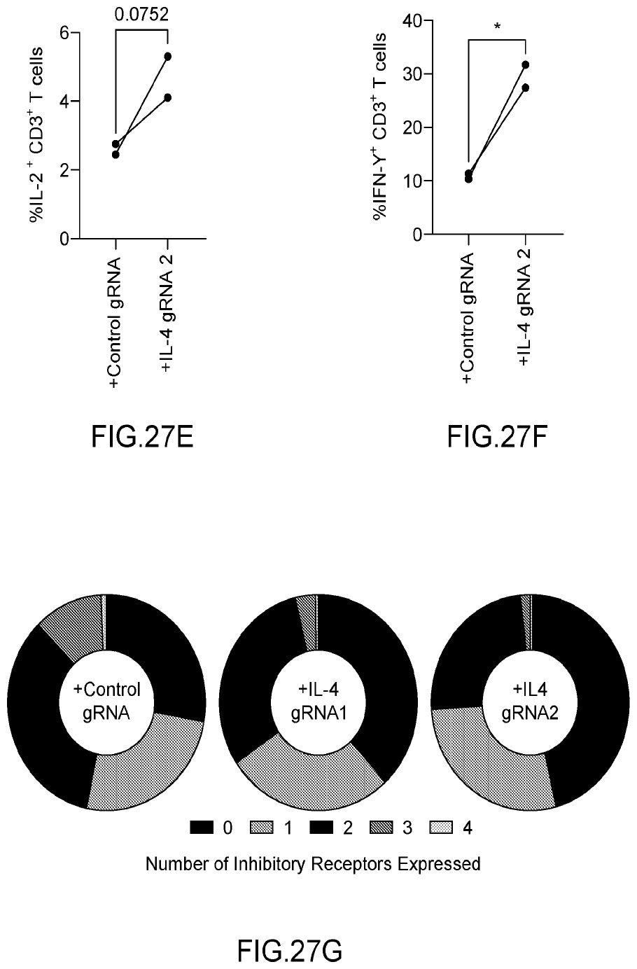

Functional changes seen after the treatment of CART19-28ζ cells with 20 ng/mL hrIL-4 did not appear to be a result of an effect of hrIL-4 on JeKo-1 tumor cells. Continuous treatment of CART19-28ζ cells with 20 ng/mL hrIL-4 resulted in an increase in the percent of CAR T cells expressing multiple inhibitory receptors by Day 15 (FIG. 23). Continuous treatment of CAR T cells with hrIL-4 still enhanced the exhaustion profile by Day 15 when the CAR T cells were exposed to cell-free stimulation by CD19 beads. This is seen by a decrease in proliferative ability (FIG. 24A), a decrease in IL-2 production (FIG. 24B), and an increase in the percent of CAR T cells expressing multiple inhibitory receptors (FIG. 24C).

### Example 6: IL-4 as a Regulator of Exhaustion in Different CAR Constructs

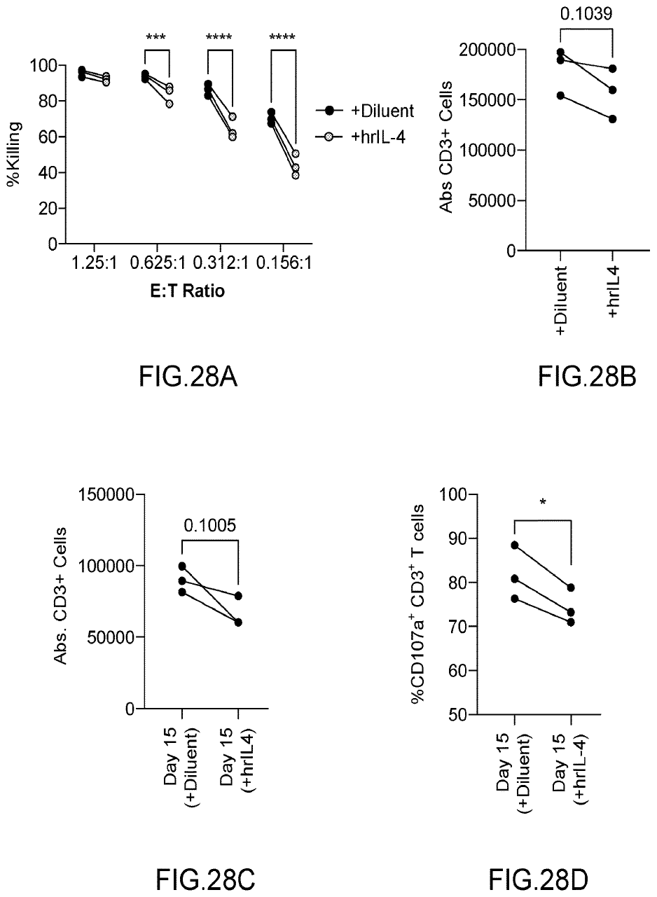

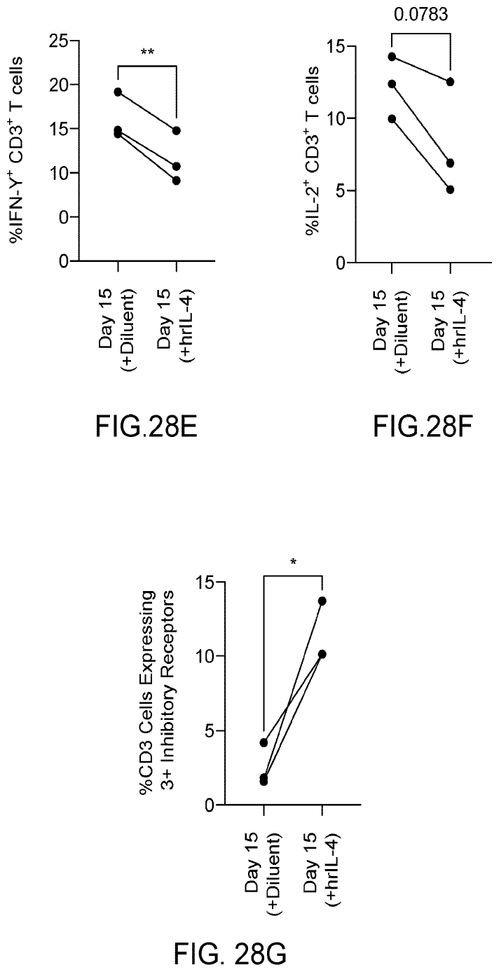

IL-4 also appears to influence the prevalence of exhaustion in CART19 cells with a 4-1BB costimulatory domain (CART19-BBζ). Treatment of CART19-BBζ cells with 20 ng/mL hrIL-4 resulted in decreased cytotoxicity (FIG. 28A) and a trend for decreased proliferation (FIG. 28B) at baseline. Upon chronic stimulation of CART19-BBQ cells in the presence of hrIL-4 as opposed to diluent, the proliferative ability decreased (FIG. 28C), the ability of CAR T cells to degranulate decreased (FIG. 28D), the production of effector cytokines such as IFN-γ and IL-2 decreased (FIGS. 28E-28F), and the percentage of CAR T cells expressing multiple inhibitory receptors increased (FIG. 28G). Together, this indicates that IL-4 can push CART19-BBC cells towards an exhausted phenotype.

### Example 7: Exemplary Sequences

(SEQ ID NO:13)
Exemplary Cas9 polypeptide sequence
MDKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDHIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

### Example 8: Treating Cancer

A human having cancer is administered CAR T cells having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells).

The administered CAR T cells having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) can target (e.g., target and destroy) cancer cells (e.g., cancer cells expressing a tumor antigen targeted by the CAR T cells) within a mammal.

### Example 9: Treating Cancer

T cells are obtained from a mammal having cancer and are engineered be CAR T cells having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells).

The CAR T cells having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) are administered back to the human. The administered CAR T cells having a reduced level of an IL-4 polypeptide (e.g., IL-4 KO CAR T cells), a reduced level of a TCF7 polypeptide (e.g., TCF7 KO CAR T cells), a reduced level of a PTPN2 polypeptide (e.g., PTPN2 KO CAR T cells), and/or a reduced level of a PTPN3 polypeptide (e.g., PTPN3 KO CAR T cells) can target (e.g., target and destroy) cancer cells (e.g., cancer cells expressing a tumor antigen targeted by the CAR T cells) within a mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A T cell having reduced susceptibility to T cell exhaustion, wherein said T cell comprises (a) a reduced level of an interleukin (IL) 4 polypeptide, a reduced level of a transcription factor 7 (TCF7) polypeptide, a reduced level of a protein tyrosine phosphatase non-receptor type 2 (PTPN2) polypeptide or a reduced level of a protein tyrosine phosphatase non-receptor type 3 (PTPN3) polypeptide, and (b) nucleic acid encoding a chimeric antigen receptor, and wherein said T cell expresses said chimeric antigen receptor.

2. The T cell of claim 1, wherein said T cell comprises a reduced level of an IL-4 polypeptide.

3-5. (canceled)

6. The T cell of claim 1, wherein said T cell comprises a reduced level of a TCF7 polypeptide.

7-9. (canceled)

10. The T cell of claim 1, wherein said T cell comprises a reduced level of a PTPN2 polypeptide.

11-13. (canceled)

14. The T cell of claim 1, wherein said T cell comprises a reduced level of a PTPN3 polypeptide.

15-17. (canceled)

18. The T cell of claim 1, wherein said chimeric antigen receptor targets a tumor-associated antigen.

19. The T cell of claim 18, wherein said tumor-associated antigen is CD19.

20. The T cell of claim 1, wherein said T cell is obtained from a human.

21-24. (canceled)

25. A method for treating a mammal having cancer, wherein said method comprises administering, to said mammal, a composition comprising a T cell having reduced susceptibility to T cell exhaustion, wherein said T cell comprises (a) a reduced level of an interleukin (IL) 4 polypeptide, a reduced level of a transcription factor 7 (TCF7) polypeptide, a reduced level of a protein tyrosine phosphatase non-receptor type 2 (PTPN2) polypeptide or a reduced level of a protein tyrosine phosphatase non-receptor type 3 (PTPN3) polypeptide, and (b) nucleic acid encoding a chimeric antigen receptor, and wherein said T cell expresses said chimeric antigen receptor.

26. The method of claim 25, wherein said mammal is identified as being in need of T cells having reduced susceptibility to CAR T cell exhaustion.

27. The method of claim 25, wherein said mammal is a human.

28. The method of claim 25, wherein said composition comprises from about 0.1×10⁶ to about 10×10⁶ of said T cells.

29. The method of claim 25, wherein said cancer is a lymphoma.

30. The method of claim 29, wherein said lymphoma is a diffuse large B cell lymphoma.

31-32. (canceled)

33. The method of claim 25, wherein said chimeric antigen receptor targets a tumor-associated antigen.

34. The method of claim 33, wherein said tumor-associated antigen is CD19.

35-37. (canceled)

38. A method for providing a mammal with CAR T cells having a reduced susceptibility to T cell exhaustion, wherein said method comprises:
(a) administering CAR T cells to said mammal, and
(b) administering an inhibitor of an IL-4 polypeptide, an inhibitor of a TCF7 polypeptide, or an inhibitor of a PTPN3 polypeptide to said mammal,
wherein said CAR T cells do not exhibit T cell exhaustion within said mammal as rapidly as comparable CAR T cells administered to a comparable mammal not administered said inhibitor of said IL-4 polypeptide, said inhibitor of said TCF7 polypeptide, and said inhibitor of said PTPN3 polypeptide.

39. The method of claim 38, wherein said mammal is identified as being in need of T cells having reduced susceptibility to CAR T cell exhaustion.

40. The method of claim 38 any one of claims 38-39, wherein said mammal is a human.

41. The method of claim 38, wherein said composition comprises from about 0.1×10⁶ to about 10×10⁶ of said T cells.

42-69. (canceled)


SEQUENCE LISTING (14 sequences)

SEQ ID NO: 1 - DNA, 20 bp, synthetic construct
  source 1..20: mol_type = other DNA
tgatatcgcacttgtgtccg

SEQ ID NO: 2 - DNA, 20 bp, synthetic construct
  source 1..20: mol_type = other DNA
caagtgcgatatcaccttac

SEQ ID NO: 3 - DNA, 20 bp, synthetic construct
  source 1..20: mol_type = other DNA
ggttcctgtcgagccgtttc

SEQ ID NO: 4 - DNA, 20 bp, synthetic construct
  source 1..20: mol_type = other DNA
gtcgctcgtgaacgagtccg

SEQ ID NO: 5 - DNA, 20 bp, synthetic construct
  source 1..20: mol_type = other DNA
cgcgctgtcgcgagaagagc

SEQ ID NO: 6 - DNA, 20 bp, synthetic construct
  source 1..20: mol_type = other DNA
cacgagcgacgacttgagct

SEQ ID NO: 7 - DNA, 20 bp, synthetic construct
  source 1..20: mol_type = other DNA
ctcttcgaactcccgctcga

SEQ ID NO: 8 - DNA, 20 bp, synthetic construct
  source 1..20: mol_type = other DNA
agttggatactcagcgtcgc

SEQ ID NO: 9 - DNA, 20 bp, synthetic construct
  source 1..20: mol_type = other DNA
ccatgactatcctcatagag

SEQ ID NO: 10 - DNA, 20 bp, synthetic construct
  source 1..20: mol_type = other DNA
acagcatgatgacgactccg

SEQ ID NO: 11 - DNA, 20 bp, synthetic construct
  source 1..20: mol_type = other DNA
ggatatggtgcacaaccacc

SEQ ID NO: 12 - DNA, 20 bp, synthetic construct
  source 1..20: mol_type = other DNA
ctgaagatggatatttgcga

SEQ ID NO: 13 - protein, 1368 aa, synthetic construct [sequence omitted]

SEQ ID NO: 14 - DNA, 20 bp, synthetic construct
  source 1..20: mol_type = other DNA
gtattactgatattggtggg